(19) United States
(12) Patent Application Publication
(10) Pub. No.: US 2025/0325454 A1
(43) Pub. Date: Oct. 23, 2025

(54) SYSTEMS AND METHODS FOR ANTI-WRINKLE FACIAL HERBAL INFUSION

(71) Applicant: Yanqun Zhong-Ardito, Blackwood, NJ (US)

(72) Inventor: Yanqun Zhong-Ardito, Blackwood, NJ (US)

(21) Appl. No.: 19/185,108

(22) Filed: Apr. 21, 2025

Related U.S. Application Data
(60) Provisional application No. 63/636,748, filed on Apr. 20, 2024.

(51) Int. Cl. A61K 8/02; A45D 44/00; A61Q 19/08

(52) U.S. Cl. CPC A61K 8/0212; A45D 44/002; A61Q 19/08; A61K 2800/5922; A61K 2800/87

(57) ABSTRACT

A dermal patch system may include a non-woven fabric backing, a reinforcement component (such as PET strips), and an adhesive layer formulated with herbal extracts or, optionally, a transparent or distinct analgesic blend. The adhesive layer may be prepared by dissolving select components in heated purified water to form a sticky solution that is later coated on the substrate, with optional release paper applied. These patches may address wrinkles by transdermally delivering a rejuvenatory herbal formula and inhibiting muscle contractions.

### REFERENCE TO RELATED APPLICATIONS

This application claims priority from U.S. provisional application Ser. No. 63/636,748, filed Apr. 20, 2024, which is incorporated by reference.

### BACKGROUND OF THE INVENTION

This application relates to a facial rejuvenation patch that includes a non-woven fabric backing layer, a layer of PET or other reinforcement strips, an adhesive layer, and a peelable release paper.

The skin, being the largest organ of the body, is exposed to various environmental factors such as sunlight, pollution, and harsh weather conditions. These factors, along with the natural aging process, can lead to the development of wrinkles and fine lines, particularly on the face. Various methods have been used to combat these signs of aging, including creams, serums, and surgical procedures. However, these methods often have limitations. For example, creams and serums may not penetrate the skin deeply enough to have a significant effect, and surgical procedures can be invasive and carry risks. Additionally, these methods may not provide a long-lasting solution, as the skin continues to age and be exposed to environmental factors. Therefore, there is a need for a non-invasive, effective, and long-lasting solution to combat the signs of aging on the skin.

### SUMMARY OF THE INVENTION

In some aspects, the techniques described herein relate to a facial rejuvenation patch, including: a. a non-woven fabric backing layer; b. a layer of PET or other reinforcement strips disposed on said backing layer; c. an adhesive layer including a mixture of herbal extracts and vitamins, including Poria cocos mushroom, Radix et Rhizoma salviae Miltiorrhizae, Rhizoma dioscorea, Polygonum cuspidatum, Angelica dahurica, Amygdalus communis Vas, Radix et Rhizoma Notogiseng, and Rhizoma ginseng, applied over said layer of PET or other reinforcement strips; and d. a peelable release paper disposed on said adhesive layer, wherein: said adhesive layer is formulated to promote transdermal delivery of said herbal extracts and vitamins to improve skin elasticity and reduce the appearance of wrinkles and fine lines when applied to the skin.

In some aspects, the techniques described herein relate to a facial rejuvenation patch, wherein said herbal extracts and vitamins include 30% purified water, 20% Poria cocos mushroom, 6% Radix et Rhizoma salviae Miltiorrhizae, 20% Rhizoma dioscorea, 6% Polygonum cuspidatum, 6% Angelica dahurica, 6% Amygdalus communis Vas, 5% Radix et Rhizoma Notogiseng, and 1% Rhizoma ginseng by weight.

In some aspects, the techniques described herein relate to a facial rejuvenation patch, wherein said herbal extracts and vitamins are naturally sticky, providing an additional layer of adhesive property to said adhesive layer.

In some aspects, the techniques described herein relate to a facial rejuvenation patch, wherein said PET or other reinforcement strips inhibit excessive muscle contractions when the patch is applied to the skin.

In some aspects, the techniques described herein relate to a facial rejuvenation patch, wherein said herb and vitamin mixture includes antioxidants vitamins A, B, D, and E, as well as Zinc.

In some aspects, the techniques described herein relate to a facial rejuvenation patch, wherein said herbal extracts further provide anti-inflammatory benefits and promote blood flow.

In some aspects, the techniques described herein relate to a facial rejuvenation patch, wherein said adhesive layer is specifically contoured to conform to various areas of the face.

In some aspects, the techniques described herein relate to a facial rejuvenation patch, wherein the patch is pre-cut into shapes specifically contoured to conform to various targeted areas of the face, such as around the eyes, forehead, or mouth, each shape designed to maximize skin contact and the effectiveness of the transdermal delivery system in those areas.

In some aspects, the techniques described herein relate to a transdermal herbal patch for facial application, consisting essentially of: a. a base structure including a non-woven fabric backing and reinforcement strips; b. a therapeutically effective amount of a herbal and vitamin composition for promoting muscle regrowth, collagen regeneration, and skin repair; and c. a contoured design shaped to maintain continuous skin contact for sustained herb and vitamin absorption, wherein: the patch delivers targeted remedial agents to facial regions prone to wrinkles and fine lines over an extended period.

In some aspects, the techniques described herein relate to a transdermal herbal patch, wherein the herbal and vitamin composition includes Poria cocos mushroom, Radix et Rhizoma salviae Miltiorrhizae, Rhizoma dioscorea, and additional herbal ingredients known for their skin-enhancing properties.

In some aspects, the techniques described herein relate to a transdermal herbal patch, wherein the reinforcement strips are composed of PET material that provides structural integrity to the patch and assists in the reduction of muscle contraction.

In some aspects, the techniques described herein relate to a transdermal herbal patch, further including a release paper allowing for secure storage and easy application of the patch.

In some aspects, the techniques described herein relate to a method for manufacturing a facial patch for reducing the appearance of wrinkles and fine lines, including: a. preparing a substrate layer by affixing reinforcement strips onto a non-woven fabric backing; b. creating an adhesive layer by dissolving a composition of Poria cocos mushroom, Radix et Rhizoma salviae Miltiorrhizae, Rhizoma dioscorea, Angelica dahurica, Amygdalus communis Vas, Radix et Rhizoma Notogiseng, and Rhizoma ginseng, and vitamins in purified water heated to a boil, to form a naturally sticky solution; c. applying the adhesive layer containing the dissolved herbal and vitamin composition onto the prepared substrate layer with the reinforcement strips; and d. covering the adhesive layer with a peelable release paper, configured for secure storage and facilitating easy application of the patch to the skin, wherein: when applied to the skin, the patch is designed to facilitate transdermal delivery of the herbal and vitamin composition, thereby improving skin elasticity and reducing the appearance of wrinkles and fine lines over an extended period.

In some aspects, the techniques described herein relate to a method, wherein the composition further includes anti-inflammatory agents and agents to promote blood flow, specifically designed to form a barrier to retain moisture in the skin, enhancing the adhesive layer's effectiveness in delivering these components transdermally.

In some aspects, the techniques described herein relate to a method, wherein the composition's specific ratio conducive to transdermal delivery efficiency is achieved by dissolving the herbal extracts and vitamins in purified water at a volume or weight ratio of 80% water to the total volume or weight of the adhesive layer, with the herbal ingredients and vitamins proportionately adjusted to their therapeutic concentrations.

In some aspects, the techniques described herein relate to a method, further including a step of designing the adhesive facial patch to prevent excessive muscle contraction, wherein the reinforcement strips are strategically placed within the substrate layer to distribute the composition evenly during application and optimize the transdermal delivery system's effectiveness.

In some aspects, the techniques described herein relate to a method, further including a wrinkle reduction regimen where the manufactured facial patch is applied to the targeted area of the skin and left in place overnight, or for at least 20 minutes to overnight wear, to ensure sustained transdermal delivery of the herbal extracts and vitamins for optimal skin rejuvenation and wrinkle reduction, as facilitated by the adhesive layer's composition and the reinforcement structure's strategic placement within the patch. The above advantages and features are of representative embodiments only, and are presented only to assist in understanding the invention. It should be understood that they are not to be considered limitations on the invention as defined by the claims. Additional features and advantages of embodiments of the invention will become apparent in the following description, from the drawings, and from the claims.

### BRIEF DESCRIPTION OF THE FIGURES

The appended figures depict certain features of the various aspects described herein and are not to be considered limiting of the scope of this disclosure.

FIG. 1 illustrates a process by which an herbal infusion anti-wrinkle adhesive facial patch may be manufactured from ingredients.

FIG. 2 illustrates various facial patch layouts which may be reconfigured across a face.

FIG. 3 illustrates facial patch system layers which may be individually manufactured and assembled.

FIG. 4 illustrates an example facial patch configuration treating wrinkles on a forehead.

FIG. 5 illustrates a manufacturing process for an herbal infusion anti-wrinkle adhesive facial patch.

FIG. 6 illustrates a process by which an adhesive layer may be synthesized.

FIG. 7 illustrates exploded-layer perspective of a therapeutic warming or cooling pain patch.

FIG. 8 illustrates a plan view of the patch of FIG. 7 in its assembled state.

FIG. 9 illustrates an example of a representative placement of a clear mechanical-support patch.

### DETAILED DESCRIPTION

Systems and techniques described herein may be used to overcome the limitations of traditional methods for addressing skin aging, the formation of wrinkles, and episodic inflammatory flare-ups. Many existing approaches rely on topical formulas that remain on the skin surface for only a short duration before being partially removed or absorbed with variable consistency. This limited contact may reduce the depth and uniformity of penetration for beneficial substances. Certain individuals seek alternative measures—often rooted in traditional Chinese medicine (TCM) or in invasive procedures—which may introduce additional complexity or discomfort in daily routines.

In some conventional implementations, fabrics or adhesive-based products may fail to deliver targeted therapies and may not provide mechanical support to areas prone to excessive muscle contractions. A single-layer fabric or cream can drift, peel prematurely, or lack structural reinforcement that would otherwise help maintain consistent contact. These observations point to a need for devices and techniques that remain in place longer, offer more consistent distribution of active ingredients, and potentially address localized muscle movements linked to skin tension, expression lines, or motion-induced flare-ups.

To address these issues, the present disclosure provides a dermal patch that may include multiple layers to facilitate comfortable adherence and reliable transdermal delivery. In certain embodiments the patch further comprises a transparent mechanical-support layer: a thin, clear reinforcement that limits undesired motion without occluding or diluting the active herbal composition beneath. The disclosed subject matter may rely on a non-woven backing, the transparent reinforcing component, and an adhesive containing beneficial extracts. This structure may be configured to reduce susceptibility to peeling or shifting while allowing an array of naturally derived compounds to remain against the skin for extended periods. Certain formulations may include vitamins, herbs, or other hydrophilic agents—many drawn from classical TCM botanical pharmacopeia—where each component may be proportioned to optimize absorption.

The disclosed subject matter may further incorporate a peelable release paper that maintains the adhesive layer in a stable condition until use. By designing various shapes or contours, the patch may conform to areas such as the forehead, corners of the eyes, or portions of the face especially prone to creasing. In some aspects, the transparent mechanical support may limit muscle movement sufficiently to reduce further stress on target regions, yet do so without impeding diffusion from the active herbal layer. Each of these features may be adaptable to different forms of dermal intervention, including cosmetic, comfort, and therapeutic applications.

An example technique may include a patch constructed with a first, non-woven fabric backing layer upon which a second, transparent reinforcing structure may be disposed. A third adhesive layer containing selected active compounds—such as Poria cocos mushroom, Radix et Rhizoma salviae Miltiorrhizae, Rhizoma dioscorea, and other TCM-sourced herbal extracts—may be applied on top of both the backing and the reinforcement. A peelable release paper may then be placed over the adhesive layer until the patch is ready for placement on the skin. In some implementations, the adhesive composition may be formulated so that it contributes both cling and therapeutic properties when worn.

Consider a scenario in which a community healthcare volunteer visits a remote clinic that sees individuals experiencing dryness, fine lines, and intermittent inflammatory flare-ups around their eyes and forehead. The volunteer may carry a supply of these dermal patches to offer a convenient, non-invasive means of providing consistent herbal nourishment. A user may remove the release paper, position the patch over the targeted region, and gently press to ensure full contact. The clear reinforcement strips may help keep the patch in place, reducing potential wrinkling or slippage as the person goes about daily tasks. Over the course of several hours, the adhesive layer may gradually deliver the herbal extracts and vitamins, promoting transdermal contact in an environment where specialized skincare services are scarce.

In this example, the user may find the patch beneficial if local conditions make frequent reapplication of topical creams difficult. When the individual completes daily activities, the patch may still remain affixed, enabling extended exposure for subtle improvements in skin elasticity and for soothing flare-ups associated with environmental stress. The volunteer may instruct the user to remove the patch at a convenient time, such as before resting in the evening or at a scheduled point of care. This process may be repeated over several days, meeting the volunteer's goal of offering a supportive measure for overall skin hydration and care in a setting where more resource-intensive procedures are less accessible. Such a scenario highlights how the system—through a dermal patch with transparent mechanical support and an active TCM-inspired herbal matrix—may provide a socially conscious application while preserving comfort and ease of use.

Illustrative embodiments of the invention are described below. The following explanation provides specific details for a thorough understanding of and enabling description for these embodiments. One skilled in the art will understand that the invention may be practiced without such details. In other examples, well-known structures and functions have not been shown or described in detail to avoid unnecessarily obscuring the description of the embodiments.

Unless the context clearly requires otherwise, throughout the description and the claims, the words comprise, comprising, and the like are to be construed in an inclusive sense as opposed to an exclusive or exhaustive sense; that is to say, in the sense of including, but not limited to. Words using the singular or plural number also include the plural or singular number respectively. Additionally, the words herein, above, below and words of similar import, when used in this application, shall refer to this application as a whole and not to any particular portions of this application. When the claims use the word or in reference to a list of two or more items, that word covers all of the following interpretations of the word: any of the items in the list, all of the items in the list and any combination of the items in the list. When the word each is used to refer to an element that was previously introduced as being at least one in number, the word each does not necessarily imply a plurality of the elements, but can also mean a singular element.

In various aspects, the present invention discloses a method for manufacturing a facial patch, designed to reduce the appearance of wrinkles and fine lines, by leveraging the therapeutic benefits of fully natural herbs, vitamins, and ingredients, herein referred to as the herbal-vitamin composition 102. This composition includes, but is not limited to, Poria cocos mushroom, Radix et Rhizoma salviae Miltiorrhizae, Rhizoma dioscorea, Angelica dahurica, Amygdalus communis Vas, Radix et Rhizoma Notogiseng, Rhizoma ginseng, and an assortment of vitamins and minerals known for their skin-enhancing properties.

In certain aspects, the vitamins, natural extracts, and ingredients (104) may be meticulously selected and prepared to ensure beneficial skin health effects. These components may be combined in a specific ratio that is conducive to transdermal delivery efficiency, wherein the sticky solution may be achieved by dissolving the composition in purified water at a volume ratio of approximately 40-90% water to the total weight of the adhesive layer, with the composition proportionately adjusted to their therapeutic concentrations. The preparation of these ingredients may involve dissolving them in purified water, heated to a boil (106), thereby forming a naturally sticky solution instrumental in creating the adhesive layer of the facial patch.

In several aspects, the process of beginning to apply the serum 108 is illustrated as a critical step in the patch manufacturing process. A brush or similar application tool, such as a large manufacturing machine for brushing efficiency, may be utilized to evenly distribute the serum across the surface of one or more substrates, which are made from a non-woven fabric substrate 110 tailored for use in facial applications. These substrates have been previously prepared by affixing reinforcement strips, made of PET, PP or glassine with fluorosilicone release, to provide structural integrity and aid in the prevention of excessive muscle contraction.

In other aspects, the manufacturing and assembly process 112 involves a meticulous layering approach. Following the application of the adhesive layer containing the healing serum, a peelable release paper is carefully affixed on top. This release paper is crucial for secure storage and facilitates easy application of the facial patch to human facial skin.

In many aspects, the facial patch is ingeniously designed to facilitate transdermal delivery of the herbal and vitamin composition, thereby improving skin elasticity and reducing the appearance of wrinkles and fine lines over an extended period. The patch's unique formulation includes anti-inflammatory agents and agents to promote blood flow, specifically designed to form a barrier to retain moisture in the skin. This enhances the effectiveness of the adhesive layer in delivering these components transdermally.

In some aspects, the sticky solution's specific ratio of ingredients is achieved by dissolving the composition in purified water at a volume ratio of approximately 40-60% water to the total weight of the adhesive layer. This ratio ensures that the composition is proportionately adjusted to their therapeutic concentrations, optimizing the patch's healing efficacy.

In various aspects, the adhesive facial patch's design also focuses on preventing excessive muscle contraction. The strategic placement of reinforcement strips within the substrate layer serves to distribute the composition evenly during application. This design choice optimizes the effectiveness of the transdermal delivery system by ensuring that the skin can evenly and continuously absorb the herbs and vitamins.

Furthermore, upon the manufacture of the facial patch, a regimen for facial skin rejuvenation is recommended. This regimen involves securing a facial patch, manufactured according to the method specified, to an area of the skin susceptible to wrinkles. Ensuring that the facial patch remains attached for an overnight period or for at least 20 minutes to overnight wear promotes an extended release and skin penetration of herbal extracts and vitamins, leading to an improvement in skin health and wrinkle reduction. This extended release is facilitated by the unique composition of the adhesive layer and the deliberate design of the facial patch's reinforcement structure.

In essence, the method for manufacturing a facial patch as disclosed herein represents a significant advancement in the field of dermatological treatments. Combining traditional knowledge of herbal medicine with modern transdermal delivery systems, this invention offers a novel and effective solution for individuals seeking to reduce the signs of aging without invasive procedures. The comprehensive approach to selecting, preparing, and applying the herbal-vitamin composition ensures that the facial patch not only delivers its active ingredients efficiently but also provides a user-friendly and non-intrusive means of improving skin health and aesthetics.

In certain aspects, the enhancement of the facial rejuvenation patch's design incorporates a deeper exploration into the choice of materials, specifically the inclusion of PET (Polyethylene Terephthalate) or other reinforcement materials, such as polypropylene (PP) or glassine with fluorosilicone release. PET, known for its durability, flexibility, and safety in contact with human skin, serves as an ideal reinforcement strip within the patch structure. This material choice not only ensures the patch's integrity during application but also aids in maintaining its position on the face throughout an extended period, such as overnight wear. Alternative materials that may serve a similar function include biodegradable polymers, which offer an eco-friendly option, or medical-grade silicones, known for their gentle contact with sensitive skin.

In addition to PET, the manufacturing and assembly process (112) may incorporate various adhesive technologies to enhance the bond between the patch and the skin without causing irritation. These may include hydrocolloid adhesives for sensitive skin types or silicone-based adhesives that allow for easy repositioning of the patch without losing adhesion quality.

The herbal-vitamin composition of the adhesive layer, rich in the therapeutic benefits of fully natural herbs, vitamins, and ingredients (102/104), is further expanded to potentially include a wider array of botanical and natural extracts. This comprehensive selection may include, but is not limited to, Atractylodes macrocephala, Bletilla striata, and asparagus for enhancing skin moisture; white peony, dandelion, and honeysuckle for their soothing and anti-inflammatory properties; Rehmannia glutinosa and Sanguisorba officinalis for promoting blood flow; and Sophora flavescens, Cortex dictamni for their antimicrobial effects.

Moreover, the inclusion of additional vitamins and natural extracts such as Retinol (Vitamin A), Vitamin C, and grape seed extract, known for their antioxidant properties, further enriches the patch's ability to combat signs of aging. These ingredients, along with the foundational elements like Poria cocos mushroom and Radix et Rhizoma salviae Miltiorrhizae, are dissolved in purified water heated to a boil 106 to create a serum that is both potent and naturally adhesive.

The adhesive layer's naturally sticky solution, formed by the boil 106 and serum 108, is meticulously brushed onto the substrates 110, ensuring an even distribution of the therapeutic serum. This layer not only serves as the medium for transdermal delivery but also incorporates the selected reinforcement materials to provide the dual benefits of skin treatment and structural support.

In several aspects, the patch's design is consciously tailored to accommodate the facial structure, allowing for targeted application to areas most susceptible to wrinkles and fine lines. The strategic placement of reinforcement strips within the non-woven fabric substrate (110) contributes to an even distribution of pressure and composition during application, further optimized by the specific ratio of ingredients conducive to transdermal delivery efficiency, as highlighted in the preparation of the sticky solution.

In several aspects, the broader spectrum of herbal and vitaminic components mentioned, coupled with the detailed structure and material choices for the patch, underscores the invention's commitment to providing a holistic, effective, and customizable approach to facial skin rejuvenation. This expanded detailing opens avenues for further innovation within the realm of dermatological treatments, positioning the Herbal Infusion Anti-Wrinkle Adhesive Facial Patch as a forefront solution in the fight against aging.

In the enhanced detailing of the invention, we delve into the precise formulation of the adhesive layer, which plays a significant role in the transdermal delivery of the rejuvenative compounds. The updated ratio of ingredients in the herbal-vitamin composition is as follows: 80% purified water serves as the solvent foundation, ensuring the herbs and vitamins are fully dissolved and activated for optimal skin absorption. The composition features 6% Poria cocos mushroom and 6% Rhizoma dioscorea, both revered for their skin-enhancing and anti-inflammatory properties.

The specifics of the formulation include 1.6% Radix et Rhizoma salviae Miltiorrhizae, known for its circulation-enhancing benefits; 1.6% Polygonum cuspidatum, which contains resveratrol, a powerful antioxidant combating aging; 1.6% Angelica dahurica, celebrated for its brightening and skin-regenerative qualities; and 1.6% Amygdalus communis Vas, more commonly known as sweet almond, revered for its hydrating and nourishing effects on the skin. Further precision in the formulation is demonstrated with 0.8% Radix et Rhizoma Notogiseng, enhancing blood circulation and promoting skin healing, alongside 0.8% Rhizoma ginseng, a potent ingredient for revitalizing and energizing the skin.

In this refined embodiment, the high concentration of purified water not only facilitates the dissolution and integration of the various herbal constituents but also ensures that the resulting mixture is of a consistency conducive to easy application. This liquid base is critical for the herbal extracts and vitamins to be effectively combined and applied as a cohesive, uniformly blended adhesive layer onto the substrate.

The process of applying the meticulously crafted serum resulting from the boil 106 onto the non-woven fabric substrate (110) via the serum (108) ensures that each ingredient is preserved in its most active form, ready to be delivered transdermally. This precision in ingredient ratios and preparation methodology underscores the invention's dedication to optimizing the therapeutic efficacy of the patch.

The inclusion of PET or equivalent reinforcement materials within the patch structure, highlighted previously, now gains further importance. Given the enriched adhesive formulation, the reinforcement layer ensures that the patch not only adheres securely to the skin throughout the extended wear time but also supports an even distribution and sustained release of the active ingredients. This ensures that the skin is continuously nourished and treated against wrinkles and fine lines, maximizing the benefits received from the high-quality natural ingredients. This inclusion of PET reinforcements worn on the face and for the purpose of skin nourishment is believed to be a novel and non-obvious contribution to enhance prior structure designs of facial beauty patches and healthcare-related systems.

Moreover, the consideration of alternative embodiments and detailed specifics of the herbal formulation allows for a degree of customization and adaptation to address various skin types and concerns. This approach not only emphasizes the versatility and innovativeness of the Herbal Infusion Anti-Wrinkle Adhesive Facial Patch but also illustrates a comprehensive understanding of dermatological health and the practical application of traditional herbal wisdom in modern skincare solutions.

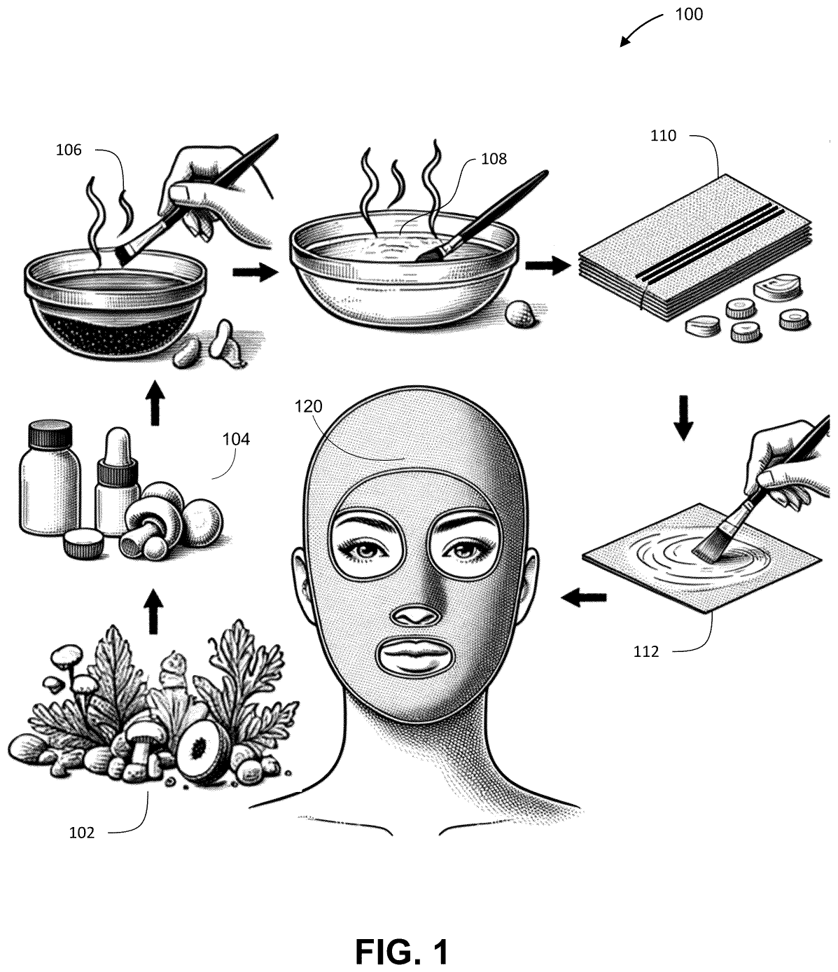

FIG. 1 illustrates a process by which an herbal infusion anti-wrinkle adhesive facial patch may be manufactured from ingredients.

In various aspects, the embodiment of the facial rejuvenation patch, as depicted in FIG. 1, innovatively incorporates multiple layers which may enhance its effectiveness and user comfort. The patch's exterior layer, crafted from a non-woven fabric, may serve as a substrate or membrane. This layer may offer structural integrity and, importantly, may be breathable and flexible, potentially conforming to the contours of the face for a seamless fit. The choice of a non-woven fabric for the exterior layer may also contribute to its hypoallergenic properties, possibly reducing the risk of skin irritation during prolonged wear.

The interior layer of substrate 110, positioned to come into direct contact with the skin, may be imbued with a special adhesive formulation derived from the processed herbal mixture. This glue, gentle yet effective, may be responsible for the transdermal delivery of the rejuvenating herbal and vitamin composition directly to the skin. It is this layer that may maintain constant contact with the facial skin, acting as the medium through which the active ingredients may be absorbed, potentially facilitating their therapeutic action in reducing wrinkles and fine lines.

Upon the completion of the manufacturing and assembly process 112, with the addition of the sticky serum, the facial patch, sized and shaped as denoted as facial system 120, may be ready for application. This final product may be crafted into various shapes and sizes to specifically target different areas of the face that are particularly prone to signs of aging, such as below the eyes, around the nose and mouth, and on the forehead. As shown, it comprehensively covers the face and much of the head, in a full wrap-style form. The ability to customize the patch's shape may ensure that users can focus on their specific concerns, whether it be crow's feet, laugh lines, or forehead wrinkles, thereby maximizing the potential effectiveness of the patch.

Further, alternative methodologies for applying the sticky serum extracted from the herbs, which acts as a natural adhesive, have been explored to enhance manufacturing ease and efficiency.

The first method, as previously described, involves applying the sticky serum atop an existing layer of glue on the non-woven fabric. This double-layered adhesive approach may ensure a strong bond with the skin and a sustained release of the active ingredients.

In an effort to simplify the manufacturing process, an alternative embodiment suggests that spraying the natural adhesive over the non-woven fabric may be much easier for manufacturers to produce. This method may allow for an even distribution of the adhesive and is considerably easier and more cost efficient for manufacturers to execute on a large scale. The spraying technique may ensure a thin, uniform layer of the herbal adhesive, promoting consistent transdermal delivery across the patch.

A novel approach to creating the adhesive involves utilizing the raw herbs directly. The herbs may be ground down to a powder and added to water that is just below boiling temperature, approximately 100 degrees Celsius. This precise temperature control may ensure the water is steamy enough to activate the herbs' adhesive properties without degrading their potency. The ground herbs may be strained, separating the solid residue from the potent, sticky serum, which may then be mixed with a common glue formulation. This method not only harnesses the natural stickiness of the herbs but also integrates it with the structural adhesive, potentially creating a robust blend that secures the patch to the skin while delivering therapeutic benefits.

These alternative embodiments for the application and formulation of the herbal adhesive offer flexibility in the patch's manufacturing, allowing for optimization based on efficiency, cost, and possible therapeutic efficacy. Whether the adhesive is brush-applied, sprayed, or mixed with common glue, each method ensures the delivery of the patch's skin-rejuvenating properties, highlighting the innovative nature of this skincare solution.

In certain aspects, the present invention relates to a wrinkle beauty product designed to offer a natural alternative to current anti-wrinkle solutions. The product is structured with a non-woven fabric backing as the first layer, over which a layer of PET or other suitable reinforcement strips is placed. Atop this layer, a unique herbal formula enriched with vitamins is applied, finalized with a peelable release paper on the topmost layer. This multi-layered construction ensures the effective delivery of the active ingredients directly to the skin, promoting better absorption and efficacy against wrinkles.

In some aspects, the purpose of this innovative wrinkle beauty product is to naturally reduce the appearance of wrinkles through a synergistic blend of herbs, vitamins, and the physical support of plastic bands. This approach stands in contrast to the prevalent use of synthetic compounds in creams and serums, which may lead to skin irritation or unwanted side effects, and invasive procedures such as Botox, which come with risks and limitations.

In various aspects, the component structure of the wrinkle beauty product leverages the skin-enhancing properties of distinct herbs combined with essential vitamins. This composition is carefully integrated into an adhesive tape, meticulously engineered to conform to different facial areas. By ensuring direct contact of the herbal and vitamin mixture with the skin, the product achieves targeted delivery of its benefits to areas affected by wrinkles, enhancing skin health and appearance with ease of use.

In other aspects, the effectiveness of the invention is further augmented by the inclusion of Poria cocos mushroom, among other potent herbs. Known for its triterpenes, polysaccharides, and essential minerals, Poria cocos demonstrates significant health benefits including immune support and enhanced cell vitality, directly contributing to the product's anti-aging, antioxidant, and skin moisturizing capabilities.

In several aspects, the preparation of the herbal formula involves dissolving the selected herbs, including Poria cocos mushroom, Radix et Rhizoma salviae Miltiorrhizae, and others, in hot boiling water. This mixture is then uniformly applied over the reinforcement layer and the non-woven fabric, creating an additional layer of natural adhesive. This process not only simplifies the product's application but also maximizes the transdermal delivery of the herbal benefits.

In many aspects, the herbal overnight patch or tape brings forth significant improvements over prior technologies by enabling the regrowth of muscle mass, collagen regeneration, and the repair of wrinkles and fine lines. The meticulously selected vitamins and herbs work in harmony to offer a superior solution for wrinkle reduction and skin rejuvenation. The inclusion of PET bands within the patch design serves a dual purpose: preventing muscle contractions that may lead to wrinkles while maintaining the patch's shape for consistent and thorough absorption of the active ingredients by the skin.

In additional aspects, the herbal composition includes Radix et Rhizoma salviae Miltiorrhizae, known for its capabilities in promoting blood circulation, removing blood stasis, nourishing blood, and calming the nerves. The chemical components of its extract, primarily fat-soluble tanshinones and water-soluble derivatives, contribute extensively to vascular health by dilating blood vessels, repairing damaged cells, and improving microcirculation. This enhances the skin's ability to regenerate and repair, playing a critical role in the wrinkle reduction process.

In further aspects, the inclusion of Polygonum cuspidatum and Angelica dahurica enriches the formula with potent antioxidant and anti-inflammatory properties. These ingredients work synergistically to promote blood flow and form a protective layer over the skin, effectively preventing dryness and improving skin elasticity. The combined effects of these components not only address surface-level wrinkles but also fortify the skin's underlying health, thereby offering a comprehensive anti-aging solution.

In yet other aspects, the formulation leverages the nutritional benefits of Amygdalus communis Vas (Almond), which is abundant in antioxidants, vitamins A, B, D, and E, and essential minerals like Zinc. Almond extract provides deep moisturization, enhances skin texture, reduces dark circles, and offers exfoliating properties. Its inclusion in the product underscores the holistic approach of the invention towards skin care, focusing on moisture retention, skin brightening, and anti-aging effects.

In still other aspects, the preparation method of the herbal formula is crucial for achieving the desired consistency and efficacy of the patch. The process involves dissolving the herbs in purified water, ensuring a potent and homogeneous mixture that can be evenly spread over the layer between the plastic bands and non-woven fabric. This method not only facilitates the optimal blending of the herbal ingredients but also enhances the natural adhesive properties of the mixture, ensuring the patch's stability and effectiveness upon application.

In many aspects, the herbal overnight patch/tape is distinguished by its profound impact on skin health, as evidenced by its ability to deliver antioxidants, regulate inflammation, and stimulate blood flow. These attributes collectively contribute to the product's capacity to maintain skin moisture, improve elasticity, and facilitate the regrowth of muscle mass and collagen. The culmination of these effects results in the visible reduction of wrinkles and fine lines.

In various aspects, the disclosure addresses the limitations of gel-based patches, which suffer from inadequate adhesion and slow release of active ingredients. By contrast, the present invention ensures a steady and effective delivery of the herbal and vitamin formula, backed by the mechanical support of PET bands. These bands not only prevent muscle contraction but also maintain the structural integrity of the patch, guaranteeing an even and continuous absorption of the nutritive components.

Upon further review, to ensure that all details of the herbs, natural ingredients, and vitamins are fully covered and integrated into the comprehensive description of the invention, let's encapsulate and elaborate on each specific component as disclosed:

In certain aspects, the invention incorporates Poria cocos mushroom, a key ingredient known for its diuretic and dampness relieving properties. Poria cocos has been highlighted for its significant role in strengthening the spleen, calming the heart, and improving the body's overall immunity. Its content of triterpenes, polysaccharides, choline, fat, lecithin, potassium, and magnesium, contributes to releasing more oxygen in the blood to supply tissue cells, thus enhancing the activity and vitality of skin cells for a moisturized and youthful appearance.

In some aspects, Radix et Rhizoma salviae Miltiorrhizae is used for its blood circulation enhancement, offering nourishing and calming effects on the nerves. Its extracts, rich in fat-soluble tanshinones and water-soluble protocatechuic aldehyde and catechin aldehyde derivatives, are crucial for vascular health and cellular repair, attributing to the efficacy of this invention in repairing damaged skin cells, improving blood vessel function, and enhancing microcirculation.

In several aspects, the formulation includes Rhizoma dioscorea, known for its effects on the digestive system, immunity, and hypoxia-resistant properties. This component plays a pivotal role in the herbal mixture, contributing to the overall wellness and enhancing the skin's resistance to environmental stressors.

In various aspects, Polygonum cuspidatum is integrated into the blend for its antioxidant, anti-cancer, and circulation-improving properties. Similarly, Angelica dahurica is added for its anti-inflammatory benefits and its capability to promote blood flow, which aids in forming a protective layer to retain skin moisture and improve skin elasticity.

In other aspects, Amygdalus communis Vas (Almond) is selected for its rich content of antioxidants and vitamins A, B, D, and E, along with Zinc, offering deep moisturization, skin brightening, and anti-aging benefits. Almond's nutritional profile supports skin health by maintaining moisture balance, repairing damaged areas, and facilitating the metabolic process for a healthier complexion.

In additional aspects, Rhizoma ginseng and Radix et Rhizoma Notogiseng are included for their comprehensive benefits on skin vitality, immune function, and anti-aging. Ginseng enhances cell vitality and nutrient supply to hair follicles, while Notoginseng contains elements that alleviate sun spots, promote skin fairness, and possess anti-freckle and blood-relieving factors.

The preparation involves a detailed method where these herbs are dissolved with hot boiling water, ensuring their active components are effectively extracted and integrated into the adhesive layer of the patch. This process not only maximizes the therapeutic potential but also enhances the adhesive property of the patch for sustained delivery and absorption of the ingredients into the skin.

In summary, the detailed incorporation of each herb, natural ingredient, and vitamin into the herbal overnight patch/tape underscores the invention's innovative approach to natural, effective skincare. The synergistic effects of these components, supported by the structural design of the patch, offer a novel solution to age-old concerns of wrinkles and skin aging, setting this invention distinctly apart from existing technologies and solutions in the skincare market.

In further aspects, this invention offers notable advantages over existing anti-wrinkle treatments by providing a direct and continuous application of beneficial herbs and vitamins to the skin. The physical and chemical structure of the patch, including PET reinforcement bands, is specifically tailored to enhance the transdermal delivery mechanism, ensuring an even, sustained absorption of the product's active ingredients. This innovative approach not only addresses the limitations of current skincare solutions but also introduces a more natural, effective method for combating the signs of aging skin.

In certain aspects, FIG. 1 (100) depicts a sequence that may illustrate how a dermal patch with an herbal-vitamin composition 102, additional ingredients 104, and a process involving boil 106, serum 108, substrate 110, manufacturing and assembly process 112, and a facial system 120 can be formed and applied. In many aspects, the herbal-vitamin composition 102 may include mushrooms, plants, or botanical extracts that may enhance skin elasticity, relieve muscle tension, or deliver analgesic benefits depending on the user's needs. The ingredients 104 may represent bottles, powders, drops, or other concentrated substances that can be selectively introduced into the patch's adhesive or serum. These ingredients 104 may include any of the extracts or vitamins disclosed for promoting transdermal delivery, such as Poria cocos mushroom, Angelica dahurica, Radix et Rhizoma Notogiseng, or ginseng, among others.

In some aspects, boil 106 depicts heating water or a water-based solution to facilitate the dissolution and integration of botanical extracts, vitamins, analgesic compounds, or optional humectants. This heated environment may help break down herbal solids, release volatile components, or ensure uniform distribution of active agents before combining them with additional mediums. For example, if a cooling analgesic blend is desired, Menthol, Menthol-type or Lygodium japonicum may be added during or after boil 106 to optimize infusion. Alternatively, if a warming analgesic blend is intended, Camphor or Cinnamon twig may be introduced at this stage, allowing thorough incorporation into the base fluid. Optional circulation-enhancing boosters such as Cyperus rotundus may be incorporated to dissipate stagnation and reinforce anti-swelling effects in chronic presentations.

In certain aspects, serum 108 results from combining the liquids and dissolved substances derived from the boil 106 step with any required stabilizers, such as Polyvinyl Pyrrolidone (PVP), Carbomers, or Hydroxyethyl Cellulose (HEC). This serum 108 may be carefully mixed until it reaches a consistency conducive to forming the third adhesive layer of a dermal patch. Depending on the chosen embodiment, the serum 108 may contain TCM-based extracts that support antibacterial, anti-inflammatory, or analgesic effects. In other variations, the serum 108 may be herb-free yet remain medically adhesive for mechanical support. In many implementations, sustained release technology or a hydrophilic macromolecule gel matrix may be integrated into serum 108 to ensure prolonged wear or controlled release of active compounds.

In other aspects, substrate 110 may represent a stack of first, non-woven, fabric backing layers, which serve as a base onto which the adhesive solution is deposited. This substrate 110 may be cut into shapes for the forehead, cheeks, or other facial regions, but in some aspects, it may also be adapted to non-facial locations such as a wrist, knee, or elbow. The substrate 110 may be formed of breathable materials or utilize optional micro-perforations to allow extended wear without trapping excessive moisture. In certain implementations, a second layer of PET or other reinforcement strips can be placed on or within substrate 110, enabling the patch to inhibit muscle contractions or limit joint movement.

In many aspects, the manufacturing and assembly process 112 may illustrate a stage in which the serum 108 is applied to substrate 110. The manufacturing and assembly process 112 may be done by brushing, spraying, or laminating the serum onto the substrate, ensuring even coverage and correct thickness. If the mixture includes a cooling analgesic blend (Embodiment 1), classical San huang-based herbs like Scutellaria baicalensis or Phellodendron amurense may be integrated for antibacterial, wound healing, or anti-inflammatory properties. If a warming analgesic blend is desired, herbs like Corydalis yanhusuo or Ligusticum chuanxiong may be present in the serum 108. In another embodiment, the serum 108 may simply be a clear mechanical adhesive for supporting an herb-free transparent or non-woven fabric backing layer with transparent adhesive that assists in limiting frowning or wrist flexion. In some aspects, the materials of the non-woven fabric patch may preserve an all-natural healing regimen.

In some implementations, the patch's backing layer may alternatively comprise a transparent aliphatic polyurethane (PU) film, thermoplastic-elastomer (TPE), or polyethylene-vinyl-acetate (EVA) film, each ranging from 12 to 75 μm in thickness. These materials may be blown or cast-extruded, corona-treated for improved adhesive compatibility, and wound into master rolls for integration. Compared to traditional non-woven fabrics, these films may offer superior optical clarity, conformability to complex facial contours, and a protective water- and microbe-resistant barrier while remaining moisture-vapor permeable-similar to Tegaderm™ or Nexcare® dressings.

In certain aspects, once the serum 108 is deposited over the substrate 110, the patch may be partially cured or dried, locking the herbal extracts (if present) into the adhesive matrix. A peelable release paper, not shown explicitly in FIG. 1, may then be placed over the adhesive surface to store the patch until application is desired. If the patch is intended primarily for the face, it may be sized or contoured in a bandage-style rectangular or half-moon shape. Alternatively, if the user aims to apply it to a wrist or elbow, a rectangular strip may be used. In some aspects, an aperture may be formed in the patch for hanging or storing, but that feature is not required. The simplest version of the patch may remain free of any aperture or extra cutout.

In many aspects, the facial system 120 indicates a region where the completed patch may ultimately be placed. This facial system 120 may correspond to areas commonly prone to wrinkles (e.g., forehead, crow's feet) or chronic muscle tension. If the user desires to reduce the appearance of lines, the patch may contain herbal extracts supportive of skin elasticity and blood flow, such as Rhizoma dioscorea or ginseng. If, instead, the user wishes to manage muscle contractions, a second layer of PET reinforcement strips may be incorporated into substrate 110 to restrict repetitive motions. FIG. 1 highlights how each step, from initial composition 102 to facial system 120, is integrated.

In other aspects, the synergy between the items labeled as ingredients 104 and the heated mixture or boil 106 may underscore how these extracts or vitamins are prepared for uniform infusion into serum 108. If the user chooses to add optional boosters such as Borneol, Frankincense, or Myrrh, those can be included in the solution at the boil 106 stage or during final mixing before the coating process in manufacturing and assembly process 112. The layering approach ensures that the chosen formula, once dried or partially cured, continues to release active compounds transdermally once placed on the intended skin area. This configuration may help address local inflammation, dryness, muscle strain, or minor surface wounds depending on the composition's focus.

In some aspects, if the user or manufacturer requires a hydrophilic macromolecule gel matrix for extended wear or deeper herb penetration, the solution forming serum 108 may incorporate cross-linking agents such as Aluminum Hydroxide or Calcium Chloride. That approach can yield a stable adhesive that adheres well to substrate 110, with minimal slippage on the skin's surface. By controlling the ratio of ingredients 104 and the temperature at boil 106, the final product can remain flexible, comfortable, and suitably adhesive for multi-hour or multi-day application. If an herbal formulation is selected, antioxidants like vitamins A, B, D, and E may help preserve beneficial properties and further enhance skin conditions.

In other embodiments, the skin-facing adhesive layer may be formulated using soft-silicone gel adhesives (such as 3M 2476P), low-tack acrylic pressure-sensitive adhesives (PSAs), or hybrid drug-in-adhesive matrices composed of acrylic or siloxane polymers. These materials are designed for hypoallergenic, latex-free use, ensuring gentle adhesion and repositionability, and can support multi-day wear without significant loss of tack or skin irritation. Application may involve slot-coating at 20-35 g m² followed by a precision die-cut release liner.

In various aspects, each step from herbal-vitamin composition 102 gathering, to ingredients 104, to the heating or boil 106, to creating serum 108, finally onto substrate 110 via manufacturing and assembly process 112, may be scaled up for commercial production or practiced on a smaller scale. This open-ended approach aligns with the broad scope of a patch that can deliver anti-wrinkle benefits, analgesic relief, muscle contraction limitation, or purely mechanical bracing. In certain embodiments, the method can remain herb-free and focus on a transparent adhesive that discreetly curbs frowning or wrist flexing, while in others, an intense TCM-based blend can be used to deliver potent revitalizing or pain-mitigating effects.

In certain aspects, the presence of facial system 120 demonstrates the final application zone without limiting the technology to facial usage alone. Once the patch is prepared and stripped of its release liner, the user presses it onto the skin, ensuring that the beneficial components from serum 108 contact the target site. Whether the objective is to reduce wrinkles, provide a cooling or warming sensation, or simply brace a muscle group, the patch's layered construction—first, non-woven backing, second layer of PET or similar reinforcement, and third adhesive mixture—facilitates a stable, comfortable bond. If an optional micro-perforated backing is desired, it may be implemented at the substrate 110 stage, channeling moisture and heat away from the skin during multi-day wear.

In many aspects, the robust functionality depicted in FIG. 1 stems directly from how each labeled element (herbal-vitamin composition 102, ingredients 104, boil 106, serum 108, substrate 110, manufacturing and assembly process 112, and facial system 120) integrates to form a comprehensive patch. If the user selects an analgesic path, the patch may offer relief for muscle aches or joint discomfort. If the user focuses on anti-aging or cosmetic goals, the patch may deliver transdermal nutrients to enhance collagen regeneration. If the user wants a simple mechanical restraint, the patch can remain herb-free yet still feature embedded reinforcement strips to limit undesired motion. Because the same essential layering approach is used, the final product remains flexible—not only physically conforming to contours but also in accommodating numerous herbal or mechanical design choices.

In certain implementations, the final patch may also be subdivided before or after manufacturing and assembly process 112, producing smaller shapes for targeted areas like crow's feet or nasolabial folds. The multi-step progression in FIG. 1 ensures that each shape can be coated thoroughly with serum 108. If cooling or warming properties are needed, they may be introduced at the boil 106 stage by adjusting the ratio of Menthol or Camphor, for example. The patch can then be packaged in single-use pouches, with or without an aperture for hanging, although the simplest patch remains free of any such opening. This open design allows an artisan to adapt the technology to many variations, from basic dryness protection to advanced muscle inhibition or TCM-based wound healing.

In other aspects, the synergy of the heated environment at boil 106 and the final bridging step of manufacturing and assembly process 112 indicates that no lumps or incomplete dissolutions of the herbal components remain in the serum 108. This uniformity ultimately leads to a stable adhesive surface that not only adheres well to the skin but also ensures consistent transdermal distribution. If the user or manufacturer chooses to incorporate a measured ratio of each herbal extract, as indicated in certain references, that ratio can remain locked in the matrix, releasing gradually once in contact with body temperature and slight perspiration. When the user gently peels away the patch, any remaining adhesive residue can often be wiped off with mild soap and water, given the water-soluble or partially hydrophilic nature of the matrix.

In many aspects, FIG. 1 thus demonstrates a straightforward yet versatile process, involving an herbal-vitamin composition 102, ingredients 104, a heated preparation at boil 106, a final serum 108, and the deposit of that serum onto substrate 110 in manufacturing and assembly process 112, culminating in a patch effectively used for a facial system 120. Whether delivering analgesic compounds, limiting muscle activity for wrinkle prevention, or providing simple mechanical support, the layering approach remains consistent. This structure clarifies how a single patch can be customized for different embodiments. The user may select the precise combination of herbs, vitamins, adhesives, or ridges to meet their personal or clinical objectives, all while preserving the general method illustrated in FIG. 1.

In many aspects, FIG. 1 thus demonstrates a straightforward yet versatile process, involving an herbal-vitamin composition 102, ingredients 104, a heated preparation at boil 106, a final serum 108, and the deposit of that serum onto substrate 110 in manufacturing and assembly process 112, culminating in a patch effectively used for a facial system 120.

It shall be noted that in some embodiments, this approach may be used across a variety of industry and technological domains, including cosmetic dermatology, sports medicine, spa-based therapeutic treatments, and consumer skincare applications. As illustrated in FIG. 1, the integration of boil 106 and serum 108 confers flexibility in selecting either a cooling analgesic blend or a warming analgesic blend, enabling localized muscle relaxation. Firstly, unlike conventional patch preparations that rely solely on chemical adhesives, the disclosed structure may incorporate transdermal nutrient delivery to enhance healing outcomes. Secondly, in some embodiments, the process may include optional micro-perforations in substrate 110, which ensures extended wear without trapping excess moisture. Furthermore, it shall be noted the synergy provided by manufacturing and assembly process 112 may preserve active herbal extracts without diminishing mechanical reinforcement from potential PET or other strips. As a result, the patch confers many technical benefits, for example improved comfort during prolonged application due to the balanced gel matrix, and a more uniform distribution of active ingredients compared to standard single-layer adhesives.

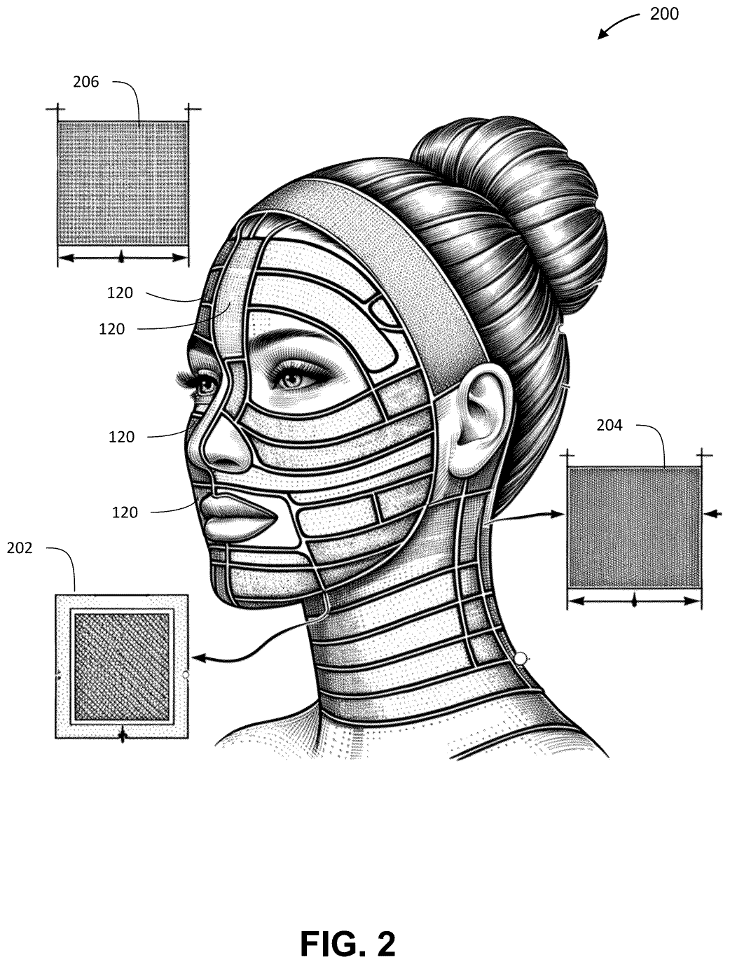

FIG. 2 illustrates various facial patch layouts which may be reconfigured across a face.

Turning now to FIG. 2, we see example embodiments of possible herbal infusion anti-wrinkle facial system configurations. These systems would have largely been manufactured according to the disclosures related to FIG. 1, and there is new variability such as the option to have two or more layers, adhesive variance across a side, and shaping and sizing for muscle targeting.

In certain aspects, the present disclosure illustrates a Variable-Adhesion Herbal System (202) designed for facial rejuvenation. This system uniquely features an increased distribution of adhesive around the edges to ensure secure placement on the skin and focused delivery of herbal infusions to targeted areas.

In some aspects, the Dual-Layer Non Woven Exterior 204 serves as the outermost shield of the Variable-Adhesion Herbal System (202). This dual-layer construction not only enhances the structural integrity of the patch but also functions as a protective barrier for the active herbal layer beneath, facilitating optimal transdermal delivery of the herbal mixture.

In various aspects, the composition includes an Advanced Pliable Bandage (206), which is crafted from a sophisticated non-woven fabric. This bandage is noteworthy for its multi-layered composition, which may include waterproof features, offering a versatile base that conforms to the contours of the face while maintaining the efficacy of the herbal infusions in various environmental conditions.

In several aspects, facial system 120 are strategically placed across different craniofacial regions to administer precise treatment where wrinkles are most prominent. These include (but are not limited to) bandages positioned at the central forehead to treat the frontalis muscle, above the eyebrows targeting the corrugator supercilii muscles, under the eyes for the orbicularis oculi muscles, and to the sides of the lips to address the orbicularis oris muscle. Each location is selected based on underlying muscle activity that contributes to wrinkle formation, providing a tailored approach to reducing the appearance of fine lines and wrinkles through targeted herbal and vitamin therapy.

In other aspects, the Herbal Infusion Anti-Wrinkle Adhesive Facial Patch represents an innovative approach to skincare, leveraging a precise blend of herbal extracts including Poria cocos mushroom, Radix et Rhizoma salviae Miltiorrhizae, Rhizoma dioscorea, Polygonum cuspidatum, Angelica dahurica, Amygdalus communis Vas, Radix et Rhizoma Notogiseng, and Rhizoma ginseng. In certain aspects, the composition of the herbal extracts is specifically designed to range from 4.8-6% for Poria cocos mushroom, 0.8-2.4% for Radix et Rhizoma salviae Miltiorrhizae, 4.8-6% for Rhizoma dioscorea, 0.8-4% for Polygonum cuspidatum, 0.8-4% for Angelica dahurica, 0.8-4% for Amygdalus communis Vas, 0.4-1.6% for Radix et Rhizoma Notogiseng, and 0.4-1.6% for Rhizoma ginseng, by weight, providing a balanced and effective formulation for enhancing skin health and reducing the appearance of wrinkles. The balance of the weight in this mixture would be approximately 80% purified water, before processing. This meticulously selected herbal composition, when combined with essential vitamins and applied via the described patch system, may significantly improve skin elasticity and diminish the visibility of fine lines and wrinkles.

In some aspects, the Herbal Infusion Anti-Wrinkle Adhesive Facial Patch may include one or more of the following ingredients: Atractylodes macrocephala, Bletilla striata, asparagus, white peony, dandelion, honeysuckle, Rehmannia glutinosa, Sanguisorba officinalis, Sophora flavescens, Cortex dictamni, Ligusticum chuanxiong, Angelica sinensis, Retinol, Vitamin C, safflower, dendrobium, astragalus, Codonopsis pilosula, Scutellaria baicalensis, Phellodendron, cucumber, rhubarb, garlic, aloe, tea, Ganoderma lucidum, Cordyceps sinensis, pearl, mother of pearl, Gynostemma pentaphyllum, licorice, Gastrodia elata, pilose antler, seaweed, lemon, turmeric, chrysanthemum, kudzu root, Polygonatum essence, Lotus leaf, Coix seed, Cayratia japonica, Aconitum leucorrhea, peony peel, mung bean, Anemarrhena asphodeloides, sesame, clove, white fruit kernel, white perilla leaf, white thistle, Polygonum multiflorum, Siraitia grosvenorii, red dates, walnuts, Gorgon euryale, ophiopogon japonicum, lotus seed, schisandra chinensis, trichosanthin, red bean, wolfberry fruit, Centella asiatica, purslane, Eucommia ulmoides, Magnolia officinalis, gallnut, Forsythia suspensa, bitter almond, Artemisia annua, grape seed, Centella asiatica, cassia seed, ginger, pumpkin seed, Sophora flavescens, corydalis, rose, mulberry Leaves, Cnidium monnieri, Smilax glabra, Kochia scoparia, Saposhnikovia divaricata, Paeonia lactiflora, Lithospermum, Rehmannia, Honeysuckle vine, Chinese prickly ash, wormwood leaf, Polygonum multiflorum, snow lotus, lavender, mint.

In many aspects, the described invention introduces a multi-faceted solution for facial rejuvenation, combining age-old herbal wisdom with modern transdermal delivery technologies. By addressing specific muscular regions responsible for wrinkle formation, this patch system offers a potent, targeted approach to skin revitalization, positioning itself as a novel contender in the realm of anti-aging skincare solutions.

In certain aspects, FIG. 2 (200) may depict a configuration in which at least one Variable-Adhesion Herbal System (202) is positioned near a human profile, and a Dual-Layer Non Woven Exterior (204) is shown at a separate enlarged view. An Advanced Pliable Bandage (206) is illustrated in another expanded detail. Each of these labeled patches may represent different ways a dermal patch may be assembled or composed, including a first, non-woven, fabric backing layer, a second layer of polyethylene terephthalate (PET) or other reinforcement strips, and a third adhesive layer containing herbal extracts and vitamins or, optionally, an herb-free mechanical formulation. In some aspects, the figure displays multiple smaller patches around the neck and possibly the forehead region, suggesting that the same fundamental layering and application technique may be implemented in more than one region of the body.

In many aspects, the Variable-Adhesion Herbal System (202) may indicate a system that integrates or modifies how strongly an adhesive composition adheres to the skin. For example, the user may choose a milder adhesive for highly sensitive facial zones or a more robust adhesive formulation for elbow, knee, or wrist applications. In some implementations, the first, non-woven, fabric backing layer and the second layer of PET or reinforcement strips may be dimensioned to produce a flexible patch that can adapt to small or large body surfaces. The figure's coarse crosshatching in the patch may be illustrative of an embedded mesh or partial weaving pattern that correlates with the second layer of PET, or it may reflect the presence of herbal particulates integrated into an adhesive matrix. In other aspects, the system may incorporate a peelable release paper (not explicitly shown in detail here) that is removed immediately prior to contacting the user's skin.

In certain aspects, the arrangement further shows a Dual-Layer Non Woven Exterior (204) having a broader, more open weave texture. This dual-layer exterior may alternatively represent an outer cover that includes or encloses the third adhesive layer. By employing a robust but breathable non-woven material, the user may achieve a patch that remains comfortable during an extended wear cycle, anywhere from a few hours to multiple days. In many implementations, the patch's non-woven layer may be impregnated with active substances or remain simply a structural layer that houses the second layer of PET or other reinforcements. For example, if the patch is used in a warming or cooling analgesic scenario, additional herbal extracts (such as Scutellaria baicalensis, Corydalis yanhusuo, Gentiana macrophylla, or Danggui) may be infused.

In several aspects, the Advanced Pliable Bandage (206) is shown as yet another variation. The condensed crosshatching around 206 may suggest a denser or more closely woven structure. It may provide an enhanced ability for partial muscle restriction, or it may be impregnated with a different ratio of hydrophilic macromolecule gels that modifies how the patch adheres or delivers active components. In certain implementations, advanced pliability may be helpful if the user wishes to place the patch over a highly contoured area, such as the side of the neck or a joint, so that the patch flexes with movement but still delivers analgesic, anti-inflammatory, or anti-wrinkle effects.

In other aspects, the large human figure in the center may display multiple smaller patches layered around the neck region and possibly across the forehead. Those smaller shapes appear as horizontally banded lines stacked along the neck, which may correspond to previously described or newly introduced reinforcement strips included within a patch. These repeated horizontal stripes may also represent multiple discrete patches worn simultaneously, each delivering a certain function (such as warming or cooling analgesia) or providing mechanical support to limit certain motions. One patch may be used for a small localized region (like around the neck to reduce muscle tension), whereas another may be broader for the upper back or scalp area.

In certain implementations, patches may represent two approaches to layering the first, non-woven, fabric backing layer and a second PET-based layer. A patch may embody an example containing a more aggressive adhesive composition or additional mechanical ridges to handle high-motion areas. Dual-Layer Non Woven Exterior 204 may reflect a gentler or more flexible dual-layer structure for sensitive locations prone to irritation. Meanwhile, Advanced Pliable Bandage 206 may also indicate a distinct composition or layering approach, possibly introducing a degradable or dissolvable layer that slowly releases analgesic or cosmetic components. Because the figure depicts them separately, it may suggest that a user or manufacturer may select which approach or layering method is most appropriate, or even unify them into a single integrated system if desired.

In many aspects, the depiction of multiple patches near the figure's head and neck area highlights that each layer may contain herbal extracts and vitamins formulated to improve skin elasticity and reduce an appearance of wrinkles and fine lines if intended for facial usage, or analgesic compounds to alleviate muscle or tendon stress if intended for trauma relief. In alternative configurations, the same layering approach may remain purely mechanical, with no herbal infusion, providing muscle contraction inhibition so that facial lines are minimized. The partial shading or crosshatching used to illustrate 202, 204, or 206 can thus correlate with a third adhesive layer containing either a warming or cooling TCM-based blend. For example, if the patch is used as a Cooling Pain Patch, Menthol may be integrated in the polymeric matrix, delivering local analgesic relief. If the patch is used in a Warming Pain Patch scenario, warming herbs such as Angelica sinensis or Cinnamomum cassia may be included to encourage blood flow.

In some aspects, the figure demonstrates how the user may place an array of patches across multiple sites simultaneously. The glimpse of squared shapes at the left suggests a close-up view of Variable-Adhesion Herbal System 202, while the right side indicates Dual-Layer Non Woven Exterior 204 with a slightly different weave or layering structure. Meanwhile, the Advanced Pliable Bandage 206 at the top may be a sample depiction of how the non-woven substrate can incorporate or bond with a thicker or more intricate reinforcement pattern. In certain implementations, each patch can be fitted with micro-perforations or optional breathability features that allow sweat or moisture to pass. If the user chooses not to include such perforations, the patch may remain fully sealed for a short-term high-adhesion application. The figure does not specifically show an aperture or opening in the patch, so if an aperture is introduced in an alternate embodiment, it may remain optional. One embodiment of the technology may further include a ring or hole to facilitate storage or secure attachment, but that is not illustrated as part of FIG. 2.

In several aspects, the partial coverage of the face in the center of FIG. 2 indicates that a forehead region patch may accompany or replace a neck region patch, demonstrating the technology's adaptability across varied anatomical curves and surface areas. If the patch is intended to deliver an herbal infusion from a composition that includes Poria cocos mushroom or Radix et Rhizoma salviae Miltiorrhizae, the user or manufacturer may vary the adhesive layer composition to ensure an appropriate concentration of vitamins, analgesic agents, and blood-flow enhancers. The same patch can incorporate reinforcement strips configured to reduce muscle contraction across the brow, consistent with earlier references to a second layer of PET or other reinforcement strips that help inhibit repeated facial muscle movements causing wrinkles.

In other aspects, referencing the flexible nature of the second layer, the Advanced Pliable Bandage 206 may use a softer PET or a polymer mesh arranged in a tessellated pattern so that the patch remains stable yet still conforms to complex shapes. The Dual-Layer Non Woven Exterior 204 may be an outer shell or may form the substrate that sits directly on the skin, depending on how the user inverts or flips the patch. The label Variable-Adhesion Herbal System 202 may simply call attention to a narrower or specialized zone of adhesive that can be modulated by adding or removing certain plasticizers or humectants. Glycerin, Propylene Glycol, or Sorbitol can be employed to modulate tackiness, while cross-linking agents may be introduced if a firmer bond is necessary. The figure's separate patches thus collectively illustrate aspects that may be used singly or in combination, fulfilling a broad range of usage scenarios.

In some aspects, the patch detail labeled 206 may also represent an embodiment wherein a user opts for a see-through film (for daytime inconspicuous wear). In some aspects, the crosshatching may merely indicate layering or structural thickness rather than color or opacity. The user may prefer to keep that patch as an advanced pliable bandage for subtle facial support, especially if repeated muscle motions on the neck or chin area lead to sagging or wrinkles. Alternatively, the patch may be adapted to the lower back or shoulders for analgesic relief, merging the same layering principles depicted in the figure.

In many aspects, the center figure with partial shading around the face and neck—where multiple horizontal stripes are drawn—may imply an arrangement in which a single user can concurrently apply multiple patches of different compositions. For example, if the user experiences both frown lines and mild shoulder tension, they may adhere a patch containing warming analgesics on the neck area, while placing a simpler mechanical or herbal-infused patch on the forehead. The user may also replace the non-woven top layer with a specialized, dual-layer approach that encloses advanced herbal solutions within micro-packets.

In certain aspects, each patch shaped like 202, 204, or 206 may further include a region set aside for brand labeling, disclaimers, or instructions. Because an aperture or loop for hanging is not shown, it remains entirely optional for a manufacturer to add. The figure likewise suggests the possibility of layering different adhesives for different edges, so that the device endures repeated bending along the neck or scalp line. The user may add antioxidants, such as vitamins A, B, D, and E, or incorporate zinc to promote faster skin repair in a cosmetic context.

In several aspects, a patch around the face or neck may reflect smaller units extracted from the same core structure as 202, 204, or 206, or may be a simpler version that omits some features. If the user wants a narrower coverage strip for the jawline specifically, that shape may still fall under a bandage-style rectangular or half-moon shape. Similarly, if the user wants to place half-moon shaped patches around the eyes, they can cut or manufacture them from the same dual-layer or advanced pliable approach. The figure illustrates the patches as the largest points of detail, but clearly the final patch can be of numerous shapes or thicknesses, so long as it retains the layered composition of a first non-woven backing, a second layer of PET or reinforcement strips, and a third adhesive layer containing (or optionally omitting) herbal extracts or vitamins.

In other aspects, the user can combine these patches in an arrangement for broad coverage, or they can rely on a single patch if minimal coverage is needed. The figure may also highlight that multiple patches can be stacked or aligned side by side if the user wants more coverage area. The figure's partial dimension arrows surrounding Advanced Pliable Bandage 206 and Dual-Layer Non Woven Exterior 204 highlight that manufacturing can scale from small squares (for localized treatments) to large sheets that may be cut down to fit a range of anatomies. If a micro-perforated approach is desired, it may be introduced into either the non-woven layer or the PET reinforcement strips, so that air flows more freely beneath the patch. That feature may or may not appear in the final product, depending on user preference or environment. The figure's minimal lines encourage the notion that each patch can be easily adapted with or without micro-perforations as needed.

In other aspects, the multiple smaller patches around the figure's neck region in FIG. 2 may represent modular segments that the user can selectively place based on muscle tension or the location of wrinkles. Each patch may be created from the same approach as 202, just subdivided. Or the user may blend the technologies from 202 and 204, layering them for a unique combination of advanced pliability plus variable adhesion. If a user modifies the patch to serve a half-moon shape for an under-chin angle or for the forehead, the same layering concept still applies. The figure's perspective also implies that the user can wear patches for cosmetic or analgesic reasons simultaneously, underlining the wide flexibility of the system.

In certain aspects, each patch may include a different ratio or concentration of herbal or vitamin extracts, depending on whether the user wants to harness the cooling analgesic approach with Menthol, Lygodium japonicum, and Yanhusuo, or a warming approach with Angelica sinensis, Guizhi, or Camphor. The same patches can also be herb-free for purely mechanical support. The crosshatching stands in for the presence of particulates or the shape of potential reinforcement strips in partial section.

In many aspects, the user may produce the patches at a manufacturing facility by first forming a spool of dual-layer material, applying adhesive or herbal infusion, then cutting them to the desired shape (square, rectangular, half-moon). The final product may be packaged with a protective release paper. Because FIG. 2 includes a large, partially shown human figure, it suggests multiple usage sites. The advanced pliable bandage approach illustrated at 206 may help reduce slippage if the user is actively moving. Meanwhile, the variable-adhesion approach at 202 may be milder for delicate skin near the scalp. The dual-layer exterior at 204 may represent a compromise solution, balancing breathability and structural support.

In other aspects, the patches can be tinted or transparent, depending on the polymer or fiber used. A user may choose a tinted patch if they prefer a subtle camouflage on their skin tone, or a transparent version for minimal visible impact. The figure remains neutral regarding color or pattern, so each patch can be produced in multiple color options. Where desired, a user may also print instructions or branding on part of the patch, though that is not shown here. If coverage across large swathes of the neck or forehead is needed, an even larger patch can be manufactured by the same layering technique. Conversely, to address smaller targeted areas, a smaller patch can be cut or formed from the same base. The figure underscores that each final patch or bandage can be placed in direct contact with the user's skin.

In many aspects, the flexible approach depicted in FIG. 2 (200) demonstrates how a user can selectively adopt the squares representing different structural or adhesive properties, or integrate them into one integrated patch product. The synergy of the first, non-woven, fabric backing layer, second PET layer, and potential herbal solution in the adhesive fosters an adaptable system for addressing wrinkles, muscle aches, or general skincare. Because the figure does not specifically depict shaped cutouts, half-moon edges, or an aperture for hanging, those remain optional features that may be introduced in a separate embodiment.

In several aspects, this figure's labeling of distinct patches, especially Variable-Adhesion Herbal System 202, Dual-Layer Non Woven Exterior 204, and Advanced Pliable Bandage 206, provides a strong visual representation of how different iterations of the same fundamental technology may appear in cross-section or texture. The user can emphasize robust adhesion with a high herbal load in one patch while employing a more mechanical, herb-free approach in another.

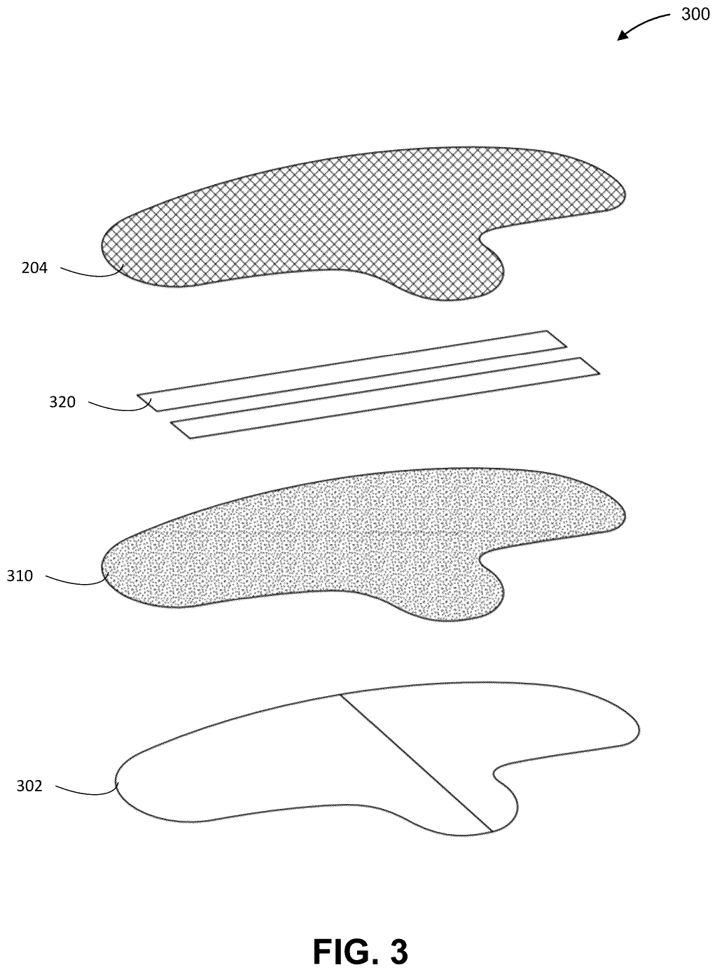

FIG. 3 illustrates facial patch system layers which may be individually manufactured and assembled.

In certain aspects, FIG. 3 illustrates the layered structure of the Herbal Infusion Anti-Wrinkle Adhesive Facial Patch, showcasing the integration of Eastern medicinal herbs with modern transdermal delivery systems. This embodiment emphasizes the patch's design to enhance skin health and reduce the visibility of wrinkles.

In some aspects, the Peelable Release Layer 302 is shown as the topmost layer of the patch, which is removed prior to application. This layer may protect the Herbal Infusion Adhesive Layer 310 from contamination and preserves its potency until the moment of use.

In various aspects, the Herbal Infusion Adhesive Layer 310 is depicted as comprising a rich mixture of vitamins, herbal extracts, and nutrients. This layer is directly responsible for the patch's therapeutic effects, including promoting muscle regrowth, collagen regeneration, and skin repair. The adhesive nature of this layer not only ensures close contact with the skin but also acts as a medium through which the active ingredients are efficiently absorbed.

In several aspects, one or more of a PET Reinforcement Set 320 are illustrated, indicating the possibility of including more strips of various sizes to enhance the structural integrity and retention of the patch on the face. The reinforcement sets may also play a role in reducing muscle contractions by providing a physical barrier that limits excessive facial movements, thereby aiding in the reduction of wrinkle formation.

In other aspects, the Dual-Layer Non Woven Exterior 204 serves as the base structure of the patch. This exterior layer not only supports the overall assembly of the patch but also contributes to its comfort and ease of wear. The non-woven fabric is selected for its breathability, flexibility, and ability to conform to the contoured design of the patch, ensuring sustained contact with the skin for extended periods.

In many aspects, alternative embodiments may involve varying the composition of the Herbal Infusion Adhesive Layer 310 to include a broader range of herbal ingredients and vitamins known for their skin-enhancing properties, such as Poria cocos mushroom, Radix et Rhizoma salviae Miltiorrhizae, Rhizoma dioscorea, and others. The specific selection of herbs and their ratios can be tailored based on the desired therapeutic outcomes, skin types, or regions of application.

In some examples, the configuration of the PET Reinforcement Set 320 may be adapted to suit different facial areas or to target specific wrinkle-prone regions more effectively. The flexibility in the layout and number of reinforcement strips allows for customization according to individual needs and preferences.

While the Peelable Release Layer 302 is essential for maintaining the patch's efficacy, innovations in material science may lead to the development of new types of release layers that offer improved preservation capabilities or that are easier to remove, enhancing the user experience.

Moreover, the potential for integrating bioactive compounds into the Herbal Infusion Adhesive Layer 310 may extend the range of therapeutic effects beyond those initially described. Considering advancements in biotechnology, encapsulation techniques such as microencapsulation or nanoencapsulation may be employed to enhance the stability and bioavailability of the active ingredients within the patch. This may allow for a controlled release mechanism, ensuring a steady delivery of the therapeutic agents over a targeted period.

In addition to, the adaptability of the PET Reinforcement Set 320 can be further exploited by incorporating smart materials capable of responding to external stimuli, such as temperature, moisture, or even the pH levels of the skin. Such materials may allow the patch to adjust its physical properties in real-time, optimizing the delivery rate of the active ingredients based on the skin's current condition or the ambient environment.

Exploring further, the development of the Peelable Release Layer 302 may also incorporate antibacterial or antimicrobial properties, which would serve to enhance the skin's health by providing an additional layer of protection against environmental contaminants. This may be particularly beneficial in patches designed for longer wear times, ensuring that the skin remains clean and reducing the risk of irritation or infection.

Regarding the Dual-Layer Non Woven Exterior 204, enhancing this component with biocompatible and eco-friendly materials may elevate the patch's appeal to environmentally conscious consumers, without compromising on its therapeutic efficacy. The exploration and use of sustainable sources for the non-woven fabric, including biodegradable or recyclable options, both aligns with global environmental goals and carbon footprint offsetting priorities, but also caters to a growing market demand for green beauty products.

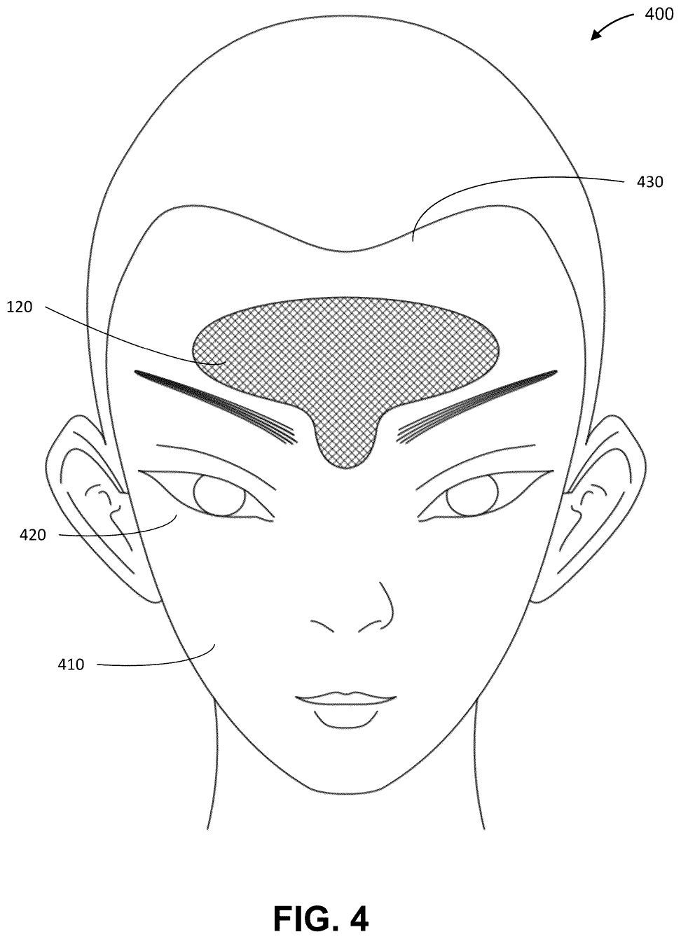

FIG. 4 illustrates an example facial patch configuration treating wrinkles on a forehead.

In certain aspects, FIG. 4 showcases the intricate design of the Herbal Infusion Anti-Wrinkle Adhesive Facial Patch, specifically engineered to address key facial regions susceptible to wrinkle formation. The patch utilizes a holistic approach, combining the ancient wisdom of Eastern medicine with cutting-edge transdermal technology to rejuvenate the skin and counteract the signs of aging.

In some aspects, the Cheek and Perioral Wrinkle Prevention Area 410 is highlighted, indicating the patch's targeted application to mitigate wrinkle formation around the cheeks and sides of the mouth and nose. This area is crucial as it involves various craniofacial muscles, including the zygomaticus major and minor, which are known to contribute to expression lines and wrinkles with repetitive movements.

In various aspects, the Periocular Wrinkle Prevention Area 420 is detailed, representing the patch's strategic placement to protect the upper cheeks and surrounding eye muscles. Given the delicate skin texture and the constant engagement of the orbicularis oculi muscle in blinking and facial expressions, this area is particularly prone to the early development of crow's feet and under-eye lines.

In several aspects, the Forehead Wrinkle Prevention Area 430 is delineated, covering the entire forehead area above the eyebrows up to the hairline. This section underscores the patch's functionality in safeguarding against horizontal forehead lines and the glabellar frown lines, attributed to the repetitive actions of the frontalis and corrugator supercilii muscles.

In other aspects, the facial system 120 embodies the invention's core, positioned to span across the forehead. This embodiment is meticulously shaped to envelop the muscles between the eyebrows as well as the forehead's wrinkle-forming muscles above the eyebrows. The design is not only aimed at providing immediate smoothing effects but also at delivering a therapeutically effective amount of herbal and vitamin composition for long-term skin health benefits.

Moreover, leveraging the understanding of all craniofacial muscles, the invention thoughtfully integrates the facial patch's herbal and vitamin composition to foster muscle regrowth, collagen regeneration, and comprehensive skin repair. The blend of Poria cocos mushroom, Radix et Rhizoma, salviae Miltiorrhizae, Rhizoma dioscorea, alongside additional potent herbal ingredients, taps into the regenerative properties of Eastern botanicals to revitalize the skin from within.

In some examples, the base structure, including a non-woven fabric backing and PET reinforcement strips, underscores the patch's commitment to comfort and efficacy. The non-woven fabric ensures breathability and flexibility, while the reinforcement strips lend structural integrity, enabling the patch to maintain continuous skin contact for sustained absorption of the remedial agents.

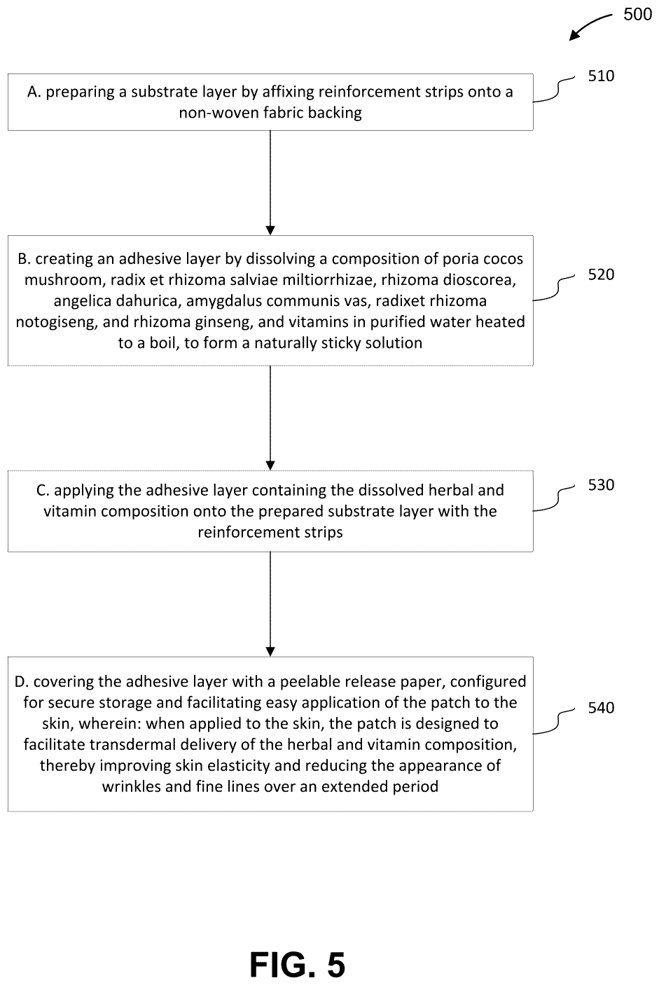

FIG. 5 illustrates a manufacturing process for an herbal infusion anti-wrinkle adhesive facial patch.

Turning now to FIG. 5, Initiating the process with Substrate Layer Preparation 510, this step involves affixing reinforcement strips onto a non-woven fabric backing, laying the groundwork for a robust and flexible foundation. The choice of non-woven fabric is pivotal, selected for its breathability and softness, ensuring that the patch can be comfortably worn for prolonged periods without causing skin irritation. This layer acts as the scaffold upon which the rest of the patch structure is built, emphasizing the importance of a durable yet pliant base that adheres smoothly to the contours of the face.

Moving to Adhesive Solution Creation 520, this phase is central to the patch's functionality, where a unique blend of herbal extracts and vitamins is prepared. The mixture includes revered ingredients such as Poria cocos mushroom, Radix et Rhizoma salviae Miltiorrhizae, Rhizoma dioscorea, Angelica Dahurica, Amygdalus communis Vas, Radix et Rhizoma Notogiseng, and Rhizoma ginseng. These are dissolved in purified water brought to a boil, resulting in a naturally sticky adhesive solution. This sticky medium is not just an adhesive but a potent carrier of therapeutic agents, optimized for effective transdermal delivery. In modern manufacturing, techniques like cold infusion or ultrasonic extraction may be explored to enhance the extraction of active compounds from the herbs, aiming to preserve their potency and maximize their skin-enhancing benefits.

In the phase of Adhesive Layer Application 530, the prepared adhesive, rich in herbal and vitamin content, is meticulously applied over the reinforced substrate layer. Ensuring a uniform coating is crucial for consistent and effective delivery of the active ingredients across the targeted skin area. Advanced manufacturing technologies such as precision coating and microencapsulation can be employed to control the release rates of active ingredients, facilitating sustained delivery over extended periods, which is critical for achieving the desired anti-aging effects.

The process is completed with Release Paper Configuration 540, where a peelable release paper is placed over the adhesive layer. This element safeguards the integrity of the therapeutic layer, maintaining the patch's efficacy until it is ready to be used. The release paper also simplifies application, ensuring the patch can be easily and securely applied to the skin. Leveraging modem materials that offer enhanced protection against environmental factors and ensure ease of peeling can further elevate the user experience, making the patch not only a therapeutic tool but also a convenient addition to skincare routines.

As the patch is applied to the skin, it is ingeniously designed to facilitate the transdermal delivery of the herbal and vitamin concoction. Employing cutting-edge technologies and leveraging the principles of skin physiology, the patch promotes skin elasticity and reduces wrinkles. Innovations in the field, such as the integration of nanotechnology for improved penetration of active ingredients, or the use of smart polymers that respond to skin's moisture levels to adjust release rates, are at the forefront of enhancing the efficacy of such transdermal systems. This approach marries ancient wisdom with contemporary science, offering a sophisticated solution to the timeless pursuit of youthful skin.

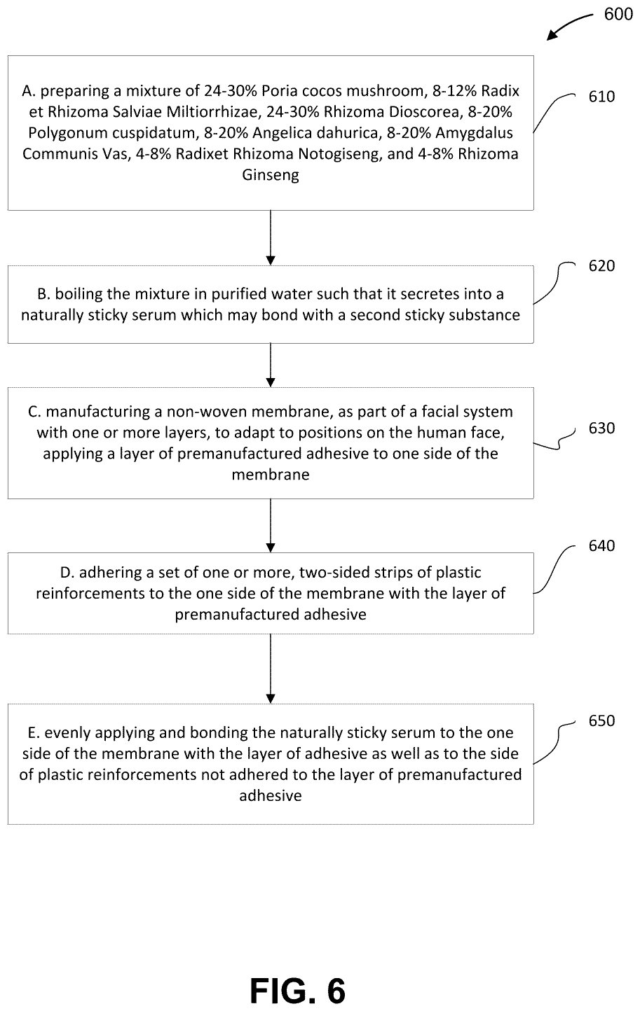

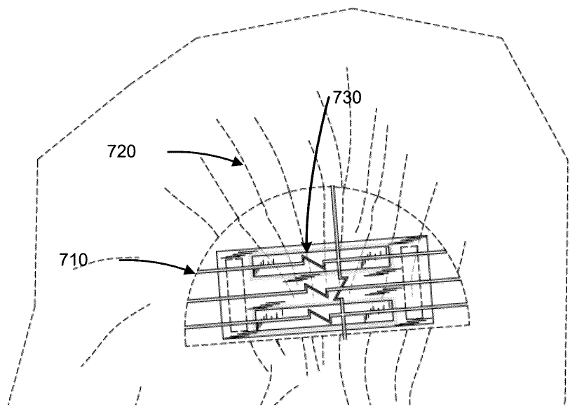

FIG. 6 illustrates a process by which an adhesive layer may be synthesized.

In several aspects, the present invention entails a meticulous method for creating an Herbal Infusion Anti-Wrinkle Adhesive Facial Patch, as illustrated in FIG. 6. The process begins with preparing a mixture 610, involving a precise formulation of herbal constituents. This includes 24-30% Poria cocos mushroom, indicative of its significant moisture retention and anti-inflammatory properties, 8-12% Radix et Rhizoma salviae Miltiorrhizae for promoting blood circulation, 24-30% Rhizoma dioscorea for its skin-soothing capabilities, 8-20% Polygonum cuspidatum known for its antioxidant effects, 8-20% Angelica dahurica for enhancing skin's resilience, 8-20% Amygdalus communis Vas which may aid in skin softening, 4-8% Radix et Rhizoma Notogiseng for promoting skin health, and 4-8% Rhizoma ginseng to energize skin cells.

In various aspects, a user may bring purified water to boiling, then add herbs into the boiling purified water, resulting in the mixture forming a naturally sticky solution or serum. This serum may bond seamlessly with a second sticky substance, creating an effective medium for transdermal delivery of the herbal benefits.

In certain aspects, manufacturing a non-woven membrane 630 is detailed as an integral part of the facial system, featuring one or more layers aptly designed to conform to various facial contours. A layer of premanufactured adhesive is applied to one side of this non-woven membrane, ensuring the patch's adherence to the skin.

In other aspects, the invention involves adhering a set of plastic reinforcements 640 to the membrane. These are two-sided strips of plastic, affixed to the membrane side already coated with a layer of premanufactured adhesive. This step is crucial for providing structural integrity to the patch, allowing it to maintain its shape and effectiveness over extended periods.

In many aspects, the process concludes with evenly applying and bonding the naturally sticky serum 650 to both the side of the membrane with the adhesive layer and to the side of plastic reinforcements not adhered to the membrane. This ensures that the herbal serum is in direct contact with the skin, promoting optimal absorption of the active ingredients for enhanced efficacy in wrinkle reduction and skin rejuvenation.

This detailed description, aligned with the technical specifications and reference numbers provided in FIG. 6, encapsulates a comprehensive approach to crafting an Herbal Infusion Anti-Wrinkle Adhesive Facial Patch. The invention's uniqueness lies in its synergetic blend of traditional herbal knowledge and modem transdermal delivery techniques, offering a novel solution for combating the signs of aging.

This interconnection between traditional herbal medicine and modern dermatological practices, supports and expands upon how the Herbal Infusion Anti-Wrinkle Adhesive Facial Patch is specifically designed to deliver targeted care to facial regions prone to wrinkles and fine lines, offering a comprehensive solution for skin rejuvenation.

Through the deliberate design and composition of its layers, the Herbal Infusion Anti-Wrinkle Adhesive Facial Patch provides a novel solution for skin rejuvenation, leveraging the therapeutic potential of herbal extracts and vitamins for the reduction of wrinkles and improvement of skin elasticity.

The Herbal Infusion Anti-Wrinkle Adhesive Facial Patch underscores the commitment to evolving the patch's design and composition in alignment with advancements in material science, biotechnology, and environmental sustainability. This detailed description, grounded in the technical expertise of natural medicinal practices and modem dermatological approaches, illuminates the multifaceted design and functionality of the Herbal Infusion Anti-Wrinkle Adhesive Facial Patch.

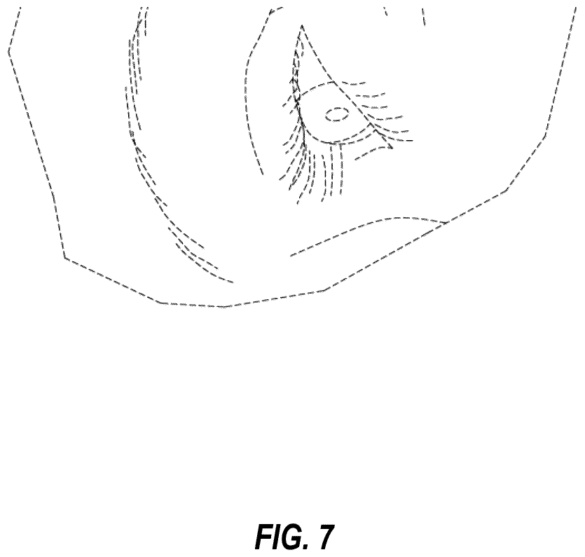

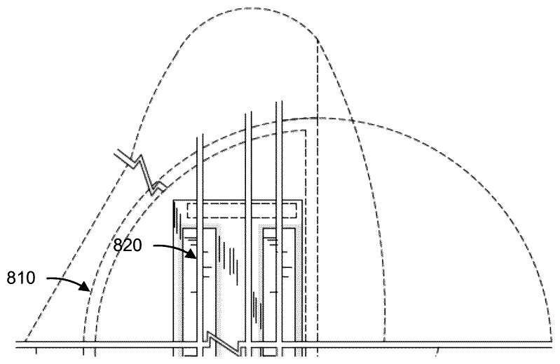

FIG. 7 illustrates exploded-layer perspective of a therapeutic warming or cooling pain patch.

In certain aspects, FIG. 7 illustrates a patch 710 disposed against a curved body region 720 in dashed outline, where the patch 710 may include multiple layers arranged to deliver herbal extracts and vitamins in some embodiments or provide mechanical support in others. In some aspects, the patch 710 may be secured to a knee, elbow, or other jointed surface to reduce tension, facilitate transdermal delivery of optional herbal formulas, and inhibit unwanted movements. For example, the primary contour of the patch 710 may be bandage-style rectangular, half-moon, or another geometry that conforms comfortably to a region requiring therapeutic support. Because the depicted curved body region 720 may represent a joint prone to swelling or muscle overextension, the patch 710 may be utilized to mitigate excessive flexion and encourage a stable range of motion. In many aspects, the materials used to form the patch 710 include a first, non-woven, fabric backing layer that supplies breathability and a second layer of polyethylene terephthalate (PET) or reinforcement strips disposed on the backing.

In some aspects, the patch 710 includes support ridges 730 that correspond to a second layer of PET strips or other reinforcement elements which may be arranged horizontally, vertically, or in an intersecting pattern. In certain aspects, these strips provide structural stiffness that may reduce muscle contractions when placed on a region prone to wrinkles or stress-induced folds, or they may help stabilize an area of the body that experiences repetitive motion. In an optional embodiment, the patch 710 rests upon the curved body region 720 in such a manner that the user experiences relief from tension when the support ridges 730 momentarily resist the movement of underlying muscles or joints. In other aspects, the user may prefer the patch 710 to remain flexible enough for comfortable daily wear, so the PET layer may be formed with partial perforations or spaced-apart ridges that yield a balance of rigidity and elasticity. For example, the patch 710 may also include micro-perforations to allow evaporation of moisture and improved ventilation, indicating that the patch may be worn for multiple days.

In some aspects, the horizontal plastic strips may be structured to counteract excessive downward pulling on the procerus muscle during intense movement of the frontalis muscle, such as during frowning. This design helps to reduce and prevent the formation of frown lines and wrinkles. The horizontal strips may be configured to resist the downward force exerted by a group of muscles—including the procerus, proxus brow, and corrugator supercilii—during frowning, further aiding in the prevention of frown-related wrinkles.

In other variations, a different shape—such as a half-moon—may be used to help prevent Crow's feet. In some implementations, the plastic strips may be positioned vertically. These vertical strips can also be designed to resist the downward pull of muscle groups including the procerus, proxus brow, and corrugator supercilii during frowning, thereby reducing the formation of frown lines and wrinkles. Since the orbicularis oculi muscle is oval-shaped, squinting not only leads to Crow's feet but may also cause wrinkles that extend toward the inner corners of the eyes, potentially making the eyes appear smaller.

In various aspects, the patch 710 may further include a third adhesive layer over the PET strips, where the adhesive layer may be infused with a combination of herbal extracts and vitamins, including, for example, Poria cocos mushroom, Radix et Rhizoma salviae Miltiorrhizae, Rhizoma dioscorea, Polygonum cuspidatum, Angelica dahurica, Amygdalus communis Vas, Radix et Rhizoma Notogiseng, and Rhizoma ginseng. In some aspects, the resulting adhesive layer may exhibit naturally sticky properties conducive to transdermal delivery, promoting improved skin elasticity and wrinkle reduction. In other aspects, a specialized mixture of vitamins A, B, D, or E, as well as minerals such as Zinc, may be incorporated to enhance antioxidant or anti-inflammatory benefits. In many implementations, the combination of herbal extracts may support blood flow and collagen production, especially advantageous if the patch 710 is used in areas subject to frequent motion that can aggravate muscle strain or joint discomfort. In certain aspects, if the aperture or shape design for removing the patch easily is not included in a base claim, then an aperture or optional cutout may be formed in the patch 710 so that a fastener or gripping point can be integrated, but such an aperture remains non-mandatory.

In some aspects, the patch 710 may be adapted for a cooling pain patch, in which the herbal infusion may feature a classical San huang base with Scutellaria baicalensis, Coptis chinensis, Phellodendron amurense, and other anti-inflammatory herbs. Such a formulation may include Menthol or a Menthol-type compound for a cooling sensation beneficial to treating acute trauma, swelling, or inflammation in a knee or elbow region. In certain aspects, the patch 710 may further contain a hydrophilic macromolecule gel matrix that uses Polyvinyl Pyrrolidone (PVP) or Carbomers to enhance sustained release of active compounds. This matrix may keep the region near the curved body region 720 cool and soothed for an extended period, reducing the need to reapply or readjust frequently. The patch 710 in such a cooling scenario benefits from the optional presence of Lygodium japonicum or Yanhusuo for additional analgesic effects. Because the support ridges 730 help to limit tension, the user may experience combined relief from muscle discomfort and mechanical support during everyday movements.

In many aspects, the same patch 710 may be adapted for a warming pain patch. In such aspects, a different herbal composition may be placed within the adhesive layer, such as Gentiana macrophylla, Angelica sinensis, and Corydalis yanhusuo. This mixture may emphasize raising local temperature and improving circulation in the curved body region 720, potentially benefiting individuals with chronic conditions or stiffness issues. In some aspects, the user may desire to incorporate Safflower, Salvia miltiorrhiza, or Camphor for an additional warming and blood-moving effect. The support ridges 730 may once again serve to reduce overstretching by providing a hybrid function of slight flex restriction and stable contact between the patch surface and the skin, enhancing the warming blend's transdermal delivery. In certain aspects, the thickness of the gel matrix or adhesive layer may be increased to store more warming botanicals, indicating that the patch 710 may be configured for a longer wear time. Optional humectants such as Glycerin or Propylene Glycol may also be used to promote moisture retention and comfort.

In other aspects, the patch 710 may incorporate a clear mechanical-support patch with no herbal composition. This embodiment relies on a medical-grade, transparent adhesive film that enables inconspicuous daytime wear. The patch 710 similarly may include the support ridges 730 to form PET ribs or ridges, which may be used to keep wrinkles from frowning. Because FIG. 7 shows horizontal striping, these ridges may be layered between a transparent top film and a lower adhesive film. In many aspects, the omission of herbal mixtures means the patch can be thinner and less noticeable than a herbal-infused variant, yet the second layer of PET or reinforcement strips still reduces motion in the curved body region 720. In certain implementations, the patch 710 with a mechanical-only approach may be used to limit undesired flexion in a wrist or prevent scowling and frowning motions on a forehead. The user may place the patch 710 upon the region requiring external support, and the resulting structure can be peeled away easily after use. Because the patch is relatively transparent, it may blend visually with the surrounding skin, encouraging consistent use during daytime activities.

In several aspects, the multi-layer arrangement of the patch 710 may be constructed by adhering the third adhesive layer to the first, non-woven, fabric backing, then introducing the PET ridges as reinforcing elements. In some exemplifications, these ridges may be partially embedded in or coated by the adhesive mixture. For example, if the user chooses a hydrophilic macromolecule gel matrix containing Glycerin, Propylene Glycol, or Sorbitol, the PET ridges may be placed at intervals or oriented in a crisscross pattern across the patch. Because the figure depicts horizontal striping for the support ridges 730, it is presumed that the orientation is optional; certain aspects may angle these strips in any direction that best matches the muscle-fiber layout or movement patterns of the curved body region 720. In some embodiments, the patch 710 may further incorporate a micro-perforated arrangement that allows the user's skin to breathe during extended hour or multi-day wear, possibly using one or more small holes distributed along the non-woven backing or through the reinforcement strips.

In certain aspects, a peelable release paper may be disposed on the outward-facing surface of the adhesive layer until the user is ready for application. Once the release layer is removed, the patch 710 can bond securely to the curved body region 720 for an extended period, whether the user aims to relieve localized inflammation, preserve range of motion in a joint, or promote transdermal nutrient delivery. FIG. 7 may be seen as illustrating an applied or in-use scenario, building upon steps in which the substrate is prepared, the composition is heated to form a naturally sticky solution, and the entire layered patch is finalized with a release paper. In many aspects, the patch 710 in FIG. 7 is sized to manage a localized region of stress or pain, but other shapes or sizes may be substituted where beneficial.

In some aspects, the patch 710 may be formulated with an alternative composition using a mixture of cooling analgesic herbs, a warming analgesic blend, or an herb-free solution. For the cooling analgesic blend, Menthol or Borneol may be included; for the warming analgesic blend, Cinnamon twig, Turmeric or a cinnamon-type ingredient may be added; for the herb-free approach, the sole functional mechanism may rely on external mechanical reinforcement offered by the PET or alternative strips. In many implementations, the patch 710 can be produced with a slightly curved geometry or flexible edges so that it may circumvent bunching or wrinkling when pressed onto a curved body region 720. Because the user's body curvature may vary significantly from one individual to another, the patch's thickness, dimension, and ratio of reinforcing strips to adhesive area may also be modified.

In certain aspects, the patch 710 also may feature optional indicators that signal overlap with a user's typical muscle or joint lines. For example, if the patch is used in the context of preventing elbow extension beyond a safe range, the support ridges 730 may be aligned with the probable area of maximum strain. Because the PET or other reinforcement strips may reduce or inhibit muscle contraction, the user may experience less tension, thus potentially promoting tissue regeneration over repeated use. In other aspects, if the user employs the patch to encourage anti-wrinkling in a region where lines are forming, the ridges may be oriented to stretch or flatten the skin slightly. In many implementations, the herbal composition fosters transdermal release to improve elasticity, and the mechanical structure helps physically discourage certain repetitive facial or bodily motions which accelerate wrinkle formation.

In some aspects, the patch 710 may incorporate a range of specific or approximate ingredient ratios describing curated percentages of Poria cocos mushroom, Rhizoma dioscorea, or others. In other aspects, the user or manufacturer may tailor the adhesive solution to incorporate advanced polypeptides or vitamins for improved collagen regeneration. Because FIG. 7 depicts the patch placed on the curved body region 720, it demonstrates that the described system may be adapted for non-facial usage. In certain aspects, the shape of patch 710 may thus be elongated to accommodate wrapping around a knee joint, or it may remain rectangular for direct application on a relatively flat region of the body.

In many implementations, the patch 710 is considered a dermal patch that may be formed by a method comprising preparing a substrate layer with or without embedded strips, dissolving the herbal extracts in purified water if chosen, applying that mixture onto the substrate, and securing a peelable release layer over top. The patch 710 for FIG. 7 may include such perforations that allow small channels for moisture evaporation, helping maintain a comfortable environment between the patch and the skin. The patch's physical structure, including the support ridges 730, remains consistent so that muscle or joint movement is appropriately limited, whether or not the herbal formulas are present.

In certain aspects, the resiliency of the patch 710 ensures that a user may engage in normal daily functions while wearing it. The horizontal striping or support ridges 730 may run across the central region of the patch to provide tension in a direction that contradicts typical bending at the curved body region 720. Because the user can choose among a cooling analgesic blend, a warming analgesic blend, or an herb-free mechanical approach, the patch 710 can adapt to numerous conditions, from acute swelling to chronic stiffness, or simply lines and wrinkles that a user wishes to manage. In some aspects, persistent use of such a patch may assist in stabilizing joint posture or facial lines over time, particularly if the composition includes anti-inflammatory or blood-circulation-boosting herbs.

In other aspects, the size of the patch 710 may be scaled to cover larger body surfaces or to address smaller areas. For example, a small patch may conform to the region behind the user's ear or around a narrower portion of the wrist, whereas a larger patch may extend over a broad region around the knee or the upper arm. In some aspects, adhesives may be formulated to last for multiple hours or days, with the user ideally replacing the patch once the effect diminishes. The patch 710 may also integrate either a single herb or multiple herbs, or remain completely free of herbal content for users seeking only mechanical tension relief.

In many aspects, FIG. 7 conveys the broad adaptability of a layered patch system, wherein the first non-woven fabric backing may contact the user's skin or face, the second PET or alternative reinforcement strips provide structural support, and the third adhesive layer may or may not carry herbal compositions. A peelable release paper can remain optional to protect the tacky surface prior to use. The figure's dashed lines around the curved body region 720 show that the patch 710 may be contoured to mitigate wrinkling or serve as a protective measure for wounds or injuries, and the horizontal striping for support ridges 730 highlights the manner in which integral ribbing structures can supply mechanical advantage.

In other aspects, a user may see synergy between the herbal chemical effect and the mechanical effect if they select the patch 710 to incorporate analgesic or anti-inflammatory blends. Because the user can peel and reapply if necessary—assuming the adhesive retains sufficient tack—there may be multiple sessions of usage over a single day or an overnight period. In some aspects, instructions for applying the patch 710 to the curved body region 720 may emphasize cleaning the skin, peeling away the release layer, and smoothing the patch from one edge to the other to remove air pockets. If micro-perforations are included, the user may avoid moisture buildup for extended wear. Ultimately, FIG. 7 suggests an example scenario of how the disclosed statements about layering, potentials for herbal infusion, mechanical reinforcement, or warming/cooling therapy can be implemented on a non-facial portion of a user's body, though the same patch structure may be equally applicable to various facial areas in other figures or embodiments.

In certain aspects, if the user chooses a hydrophilic macromolecule gel matrix containing Glycerin, Propylene Glycol, or Sorbitol, the PET ridges might be placed at intervals or oriented in a crisscross pattern across the patch. It shall be noted that in some embodiments, additional materials may be selected from one or more of the following: Atractylodes macrocephala, Bletilla striata, asparagus, white peony, dandelion, honeysuckle, Rehmannia glutinosa, Sanguisorba officinalis, Sophora flavescens, Cortex dictamni, ligusticum chuanxiong, Angelica sinensis, Retinol, Vitamin C, safflower, dendrobium, astragalus, Codonopsis pilosula, Scutellaria baicalensis, Phellodendron, cucumber, rhubarb, garlic, aloe, tea, Ganoderma lucidum, Cordyceps sinensis, pearl, mother of pearl, Gynostemma pentaphyllum, licorice, Gastrodia elata, pilose antler, seaweed, lemon, turmeric, chrysanthemum, kudzu root, polygonatum essence, Lotus leaf, Coix seed, Cayratia japonica, Aconitum leucorrhea, peony peel, mung bean, Anemarrhena asphodeloides, sesame, clove, white fruit kernel, white perilla leaf, white thistle, polygonum multiflorum, Siraitia grosvenorii, red dates, walnuts, Gorgon euryale, Ophiopogon japonicum, lotus seed, Schisandra chinensis, trichosanthin, red bean, wolfberry fruit, Centella asiatica, purslane, Eucommia ulmoides, Magnolia officinalis, gallnut, Forsythia suspensa, bitter almond, Artemisia annua, grape seed, Centella asiatica, cassia seed, ginger, pumpkin seed, Sophora flavescens, corydalis, rose, mulberry Leaves, Cnidium monnieri, Smilax glabra, Kochia scoparia, Saposhnikovia divaricata, Paeonia lactiflora, Lithospermum, Rehmannia, Honeysuckle vine, Chinese prickly ash, wormwood leaf, Polygonum multiflorum, snow lotus, lavender, and mint. As illustrated in FIG. 7, these components may be blended into the adhesive or gel matrix to provide additional anti-inflammatory, anti-oxidant, or other skin-enhancing benefits, thereby broadening the range of embodiments for the patch 710 and facilitating an even more comprehensive layering approach around the curved body region 720.

The patch 710 may incorporate a range of specific or approximate ingredient ratios that correspond to one or more claims describing curated percentages of Poria cocos mushroom, Rhizoma dioscorea, or others. In some embodiments, the cost-effective manufacturing of the patch 710 with support ridges 730 can be achieved by roll-to-roll processing, wherein a continuous sheet of non-woven substrate and PET or equivalent reinforcement material is fed through stations that apply the herbal-infused adhesive sequentially. This method may substantially reduce labor and fabrication time, allowing large-scale production with minimal waste. Furthermore, the user or manufacturer can adopt modular injection nozzles or brush heads during the assembly line to introduce selectively distinct serums, each containing different herbal or vitamin formulations targeting specific muscle groups or skin conditions. Such an arrangement results in many technical benefits over conventional single-pass printing techniques. Firstly, unlike conventional approaches that barely manage uniform layering on curved surfaces, roll-to-roll or modular stations can incrementally build multiple thin coats, ensuring that the final patch remains flexible and durable. Secondly, in some embodiments, the manufacturing steps may be reconfigured to insert an additional layer of reflective foil or breathable membrane underneath the PET ridges 730, thereby enabling temperature regulation or moisture control while still conforming to the curved body region 720.

In certain aspects, the resiliency of the patch 710 ensures that a user may engage in normal daily functions while wearing it. It shall be noted that once assembled, the patch 710 can be sterilized by UV or low-heat exposure before packaging to maintain hygiene standards and quality assurance. As illustrated in FIG. 7, the combination of mechanical reinforcement through ridges 730 and targeted botanical extracts ensures the patch may be used across a variety of industry and technological domains, such as sports medicine, physical therapy, integrated healthcare clinics, and even consumer wellness markets. Moreover, the user or caregiver can replace or recycle each patch after a certain wear period, as the chosen non-woven fabric and PET-based skeletal structure may be disassembled or repurposed, aligning with environmentally-conscious manufacturing goals.

In certain aspects, if the user chooses a hydrophilic macromolecule gel matrix containing Glycerin, Propylene Glycol, or Sorbitol, the PET ridges might be placed at intervals or oriented in a crisscross pattern across the patch. In some embodiments, a vacuum-assisted infusion method may be used to draw specialized active compounds and polymeric binders deeply into the non-woven backing prior to layering the reinforcement ridges. This approach can help achieve a more homogeneous composition across the patch, thereby streamlining the steps of applying or curing adhesive solutions. Integrating vacuum-assisted infusion may also reduce reliance on multiple brush-on coatings, minimizing material consumption and further lowering the production cost. Furthermore, automated sensors along the manufacturing line can measure thickness variations in real time, ensuring that the patch maintains uniform layers and delivers consistent performance in a wide range of temperature and humidity conditions.

The patch 710 may incorporate a range of specific or approximate ingredient ratios that correspond to one or more claims describing curated percentages of Poria cocos mushroom, Rhizoma dioscorea, or others.

It shall be noted that in some embodiments, a modular layering station may be positioned after the reinforcement step, wherein the user or manufacturer can selectively insert advanced sealing membranes or micro-porous films to fine-tune vapor permeability. For example, a microfleece overlay might incorporate corrosion-resistant metallic fibers that serve as reflective surfaces, directing heat back toward the user's skin if a warming analgesic approach is desired. Conversely, a thin polymer mesh could promote cooling in high-exertion environments, such as athletic training or physical therapy scenarios. Such add-on membranes further broaden the scope of potential end uses for the patch 710, while preserving mechanical support from the ridges 730.

In certain aspects, the resiliency of the patch 710 ensures that a user may engage in normal daily functions while wearing it.

In various embodiments, a multi-stage assembly line may employ inline splicing, so separate material rolls—one carrying the main herbal or vitamin adhesive coating, another bearing the PET reinforcement—merge seamlessly into a single continuous sheet. Subsequently, die cutters or laser ablation systems may shape each patch to match anatomically curved surfaces akin to the curved body region 720. This continuous flow design may offer cost-effective and time-efficient production by reducing material waste and simplifying inventory management for different patch configurations. As the manufacturing steps can be readily scaled, both small clinical batches and high-volume consumer products can be produced with minimal tooling changes, ultimately enhancing the versatility of the disclosed technology.

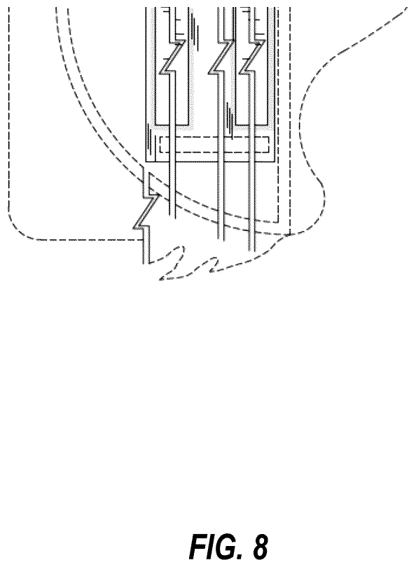

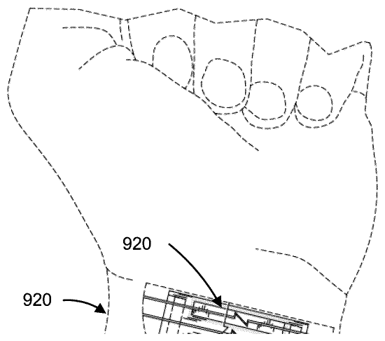

FIG. 8 illustrates a plan view of the patch of FIG. 7 in its assembled state.

In certain aspects, FIG. 8 depicts a patch 810 that may be placed on a curved or generally contoured body region, with multiple support ridges 820 oriented vertically through the patch to provide reinforcement or structural rigidity. In various aspects, the patch 810 includes a first, non-woven, fabric backing layer that may serve to cushion the skin and supply a breathable substrate. In some aspects, a second layer of polyethylene terephthalate (PET) or other reinforcement strips may be disposed on this backing layer, visible in FIG. 8 as elongated ridges that may be aligned with the user's muscle or joint lines for inhibiting excessive movement. The patch 810 may further incorporate a third adhesive layer that is optionally infused with herbal extracts and vitamins, or substituted for an herb-free formulation, depending on the user's preference or therapeutic goals.

In certain aspects, the patch 810 is shown in an upright orientation with the support ridges 820 extending from near the top to near the bottom of the patch. Because the figure includes dashed outlines of a body region behind the patch 810, it indicates that the patch may be placed on a portion of the user's face or neck, or on a non-facial site such as a knee or elbow. In many implementations, the vertical orientation of the support ridges 820 may help mitigate motion along that axis, ensuring that muscle contraction or joint flexion is partly constrained. In other aspects, these ridges may also be angled or arranged in a crisscross pattern, though FIG. 8 specifically shows them as vertical lines. The user may keep these strips partially spaced or fully adjoining, dependent upon whether greater or lesser rigidity is desired.

In some aspects, the patch 810 includes a peelable release paper (not shown in FIG. 8) that would remain on the outer adhesive surface prior to application. Once the patch is positioned, the release paper may be removed to expose a tacky or semi-tacky interface that contacts the user's skin. The adhesive layer, in many implementations, may be blended from a mixture that includes purified water and certain optional active agents, such as Poria cocos mushroom, Radix et Rhizoma salviae Miltiorrhizae, Rhizoma dioscorea, Angelica dahurica, or ginseng, among others. In some aspects, the ratio of these herbal extracts and vitamins may be selected for improved transdermal delivery, which can help reduce fine lines and wrinkles or supply anti-inflammatory benefits to an area prone to joint strain.

In other aspects, if the user desires analgesic or cooling action, the patch 810 may be formulated in alignment with Embodiment 1 (the cooling pain patch), incorporating a classical San huang base (Scutellaria baicalensis, Coptis chinensis, Phellodendron amurense) supplemented by Lygodium japonicum, Yanhusuo, and Menthol. In some implementations, this approach provides a soothing, cooling sensation while also delivering antibacterial and wound-healing properties to the region behind the patch. The presence of Menthol may be beneficial for users experiencing itching or mild swelling, while the support ridges 820 may help limit repetitive motion that aggravates the site. Similarly, the patch 810 may be adapted for a warming analgesic formula, where ingredients like Angelica sinensis and Corydalis yanhusuo promote increased blood flow and pain relief.

In several aspects, the patch 810 may be entirely transparent and free of herbal components. In such an herb-free mechanical-support patch, or transparent or non-woven fabric patch, a medical-grade clear adhesive layer by itself may provide outward pressure, and the PET reinforcement layer (shown in FIG. 8 as support ridges 820) may reduce scowling lines, wrist flexion, or other undesired movements. Because FIG. 8 demonstrates vertical ridges, a user may select that orientation to address particular muscle planes. For example, if a user is trying to limit up-and-down movement of the brow or jaw area, the vertical ridges may hamper that muscle extension. If the user instead needed to limit side-to-side motion, a different orientation of ridges may be used, though not portrayed in FIG. 8.

In some aspects, the patch 810 may further incorporate a micro-perforated structure in the first, non-woven, fabric backing layer. Although not explicitly shown, such perforations may allow air circulation across the covered region, potentially useful for multi-day wear. If the user chooses a multi-day approach, the patch's adhesive formula may be prepared to remain effective over 24 hours or more. Optional humectants like Glycerin, Sorbitol, or Propylene Glycol in a hydrophilic macromolecule gel matrix may sustain moisture levels near the skin, promoting comfortable, extended usage. Meanwhile, the user may appreciate the gentle mechanical restraining effect from the support ridges 820, especially for a joint or muscle region that may otherwise be strained by everyday motions.

In other aspects, the patch 810 may be contoured to a bandage-style rectangular or half-moon shape (not specifically depicted in FIG. 8) if the user's region of concern is around the eyes or mouth. Because FIG. 8 shows a generally rectangular patch with distinct corners, it is presumed that this patch is designed for a more linear alignment along a user's limb or face. If an aperture is desired—for example, to hang or store the patch in sterile packaging—such an aperture may be introduced in an upper or lower corner of the patch. However, it remains optional, as the simplest version of the patch design does not require it. In certain aspects, the patch orientation depicted in FIG. 8 may be reversed or tilted in final usage, allowing the support ridges 820 to align precisely with the user's muscle orientation.

In many implementations, the patch 810 is manufactured by first preparing a substrate layer (the non-woven fabric backing), then affixing or embedding the reinforcement strips that form the support ridges 820. A separate process step may then involve dissolving or mixing the desired herbal extracts and vitamins in purified water, heated to form a naturally sticky solution. This solution may be coated over the substrate and the ridges, creating a unified patch. A peelable release layer may be applied last, preserving the adhesive surface until the user is ready to apply the patch. Because the figure reveals distinct lines behind the patch, it suggests that the underlying body area is curved, indicating the patch can bend or flex to maintain contact with the skin. The ridges 820 may remain relatively stiff in their own plane, thus inhibiting unwanted expansions or contractions of muscle beneath.

In certain aspects, the patch 810 may incorporate one or more crosslinking agents (e.g., aluminum hydroxide, calcium chloride) within the gel matrix to produce a steady state of gel viscosity and consistent slow release of herbal or analgesic components. This is especially beneficial for extended-wear scenarios, as it may allow the solution to remain active for hours or even days. The user may adapt the ratio of extracts, the thickness of the PET layer, or the size of the support ridges 820 to customize the patch performance. For example, a thicker or more robust arrangement of ridges may be beneficial for a high-stress region like the knee or back, while a thinner, flexible arrangement may be better suited for facial or neck usage.

In some aspects, the patch 810 shown in FIG. 8 may be cut or shaped during production to specifically conform to areas of the human face that experience frequent wrinkling, such as the nasolabial region or marionette lines along the mouth. The vertical ridges 820 may then help flatten or resist these wrinkle-forming motions. If the user desires a purely cosmetic effect, the third adhesive layer may be loaded with vitamins A, B, D, E, and Zinc, or with a TCM blend that focuses on increasing collagen turnover and blood circulation. Alternatively, if the user primarily needs mechanical support for muscle or joint control (for example, for carpal tunnel syndrome), the patch 810 may remain herb-free or incorporate only analgesic blends for local pain reduction.

In many implementations, the vertical support ridges 820 seen in FIG. 8 can be made from PET film, polymer-based stripes, or other rigid or semi-rigid components. In certain aspects, they may be manufactured with partial perforations to maintain breathability. If a user wants additional breathability across the entire patch surface, then micro-perforations in the non-woven backing or the PET itself may be introduced. The figure also shows that these ridges appear inside the area defined by the patch 810, indicating that the ridges are not separate items placed on top but rather integrated into an internal layer. That approach is consistent with language referencing PET or other reinforcement strips disposed on the backing layer. In some aspects, the user may prefer to deposit the third adhesive layer over these ridges so that the active herbal mixture or mechanical adhesive encloses them.

In certain embodiments, the patch 810 with support ridges 820 can feature an additional partial-laminate or protective layer on top of the ridges to keep them from direct contact with external surfaces. This top laminate may be optional. In some aspects, a partial laminate can reduce the friction or snagging if the user wears the patch under clothing. Alternatively, if the user wants maximum breathability and minimal layering, the standard approach (with only the non-woven backing, PET reinforcements, adhesive composition, and release paper) may be employed.

In several aspects, the patch 810 may be contoured for a narrower or more elongated shape if the user's target region is, for example, the lower forearm or near the Achilles tendon. The figure suggests a more rectangular aspect ratio, but the same fundamental structure can be scaled up or down. In some implementations, the patch may measure only a few centimeters wide for small facial applications, whereas larger patches may be many centimeters wide for broader surfaces. The consistency in design means that the user always benefits from the synergy between the first, non-woven, fabric backing, the second layer of PET reinforcement, the optional herbal or analgesic adhesive matrix, and the protective release layer.

In many aspects, an internal hydrophilic macromolecule gel matrix may anchor the support ridges 820 in place, ensuring that they do not shift or become dislodged during normal movement. The gel matrix may also incorporate humectants to keep the user's skin hydrated. In certain aspects, if the user specifically wants a single-patch approach for overnight usage, the patch 810 can be worn for at least eight hours, delivering extended benefits. If the user is uncertain about needing herbal extracts, they may rely solely on the mechanical effect of the ridges 820. Because the invention contemplates a range of compositions and patch configurations, FIG. 8 simply highlights the vertical arrangement of the ridges in a rectangular patch layout.

In some aspects, an optional aperture may be introduced in the top edge of patch 810 if a manufacturer wishes to facilitate storage by hanging the patches in a clinic or store environment. In some aspects, the user or manufacturer may choose to omit it. In certain scenarios, leaving the patch as a sealed rectangular or half-moon shape may be simpler and provide a more uniform coverage area. The dashed lines in FIG. 8 that encircle or partially envelope patch 810 suggest that the patch may be used around the user's upper torso or possibly near the neck area, though an exactly defined body region is not specified.

In various aspects, the vertical lines of support ridges 820 can be considered a second layer of PET or reinforcement strips. By limiting muscle or joint extension along the vertical axis, the patch 810 may effectively curb certain repetitive stress motions. For example, if placed near the shoulders or upper arms, the user may experience a gentle pullback or tension as soon as they exceed normal extension. If placed on the face, these ridges may reduce upward or downward tugging on the forehead, nasolabial region, or lower jaw. The adhesive, whether herbal or purely mechanical, ensures consistent contact with the skin, thereby enabling transdermal nutrient delivery if needed. If the user chooses to incorporate one of the TCM-based analgesic blends (cooling or warming), the ratio and composition of herbs may be adapted to address inflammation, stiffness, or healing deficits. Meanwhile, the mechanical synergy of the ridges fosters partial immobilization and helps the user rest or move with caution.

In certain embodiments, the structure of patch 810 is produced using a method that includes preparing a substrate, affixing plastic reinforcements as the second layer, dissolving the herbal extracts and vitamins if applicable, applying that solution, and covering it with a peelable release paper. The final item, once removed from packaging, is placed on the user's body as indicated in FIG. 8. In some aspects, additional steps like doping the adhesive layer with analgesic or antiangiogenic agents may be performed if a specialized therapy is required.

In many implementations, the height or thickness of the support ridges 820 can be selected based on user comfort. A lighter reinforcement may be used if the user only wants mild posture correction or subtle anti-wrinkling support. A stiffer or thicker reinforcement may be employed for more significant stabilization. The same approach applies to adhesives: some versions can be short-wear for daytime, enabling easy removal and reattachment if the user wants to wash or reapply a patch, while others may be formulated with a stronger grip for overnight or multi-day adherence.

In some aspects, the figure emphasizes that the ridges 820 run the entire vertical length of patch 810, though partial or segmented ridges may be used in other embodiments. For example, segmented ridges may enable pivot points to accommodate complex joint shapes like a knee or elbow. The user or manufacturer may decide how many ridges to integrate, such that only a second layer of PET or reinforcement strips may be disposed on the backing layer to reduce or inhibit muscle contractions or joint flexion. Because the invention contemplates usage on wrists, back, or other non-facial regions, FIG. 8 provides a generalized representation of how the patch may appear once aligned with the user's anatomy.

In certain aspects, the patch 810 may also be shaped and sized to conform to narrower facial zones, such as the chin or jawline, by trimming or custom cutting. The user may trim around the corners without compromising the ridges 820, as the portion containing the PET or other strips would remain intact. That way, individuals can adapt the patch to their specific lines or tension points if they desire a more tailored fit. If an initial patch is half-moon shaped, the ridges may follow the arc of the patch, though FIG. 8 highlights a rectangular shape with essentially straight ridges.

In many aspects, the synergy between the ridges 820 and the adhesive layer may be enhanced if the user employs a hydrophilic macromolecule gel matrix to anchor everything together. These gel matrices can ensure even distribution of herbal or vitamin content if used, or they can simply keep the mechanical patch stable against sweat or water. If the user chooses to incorporate optional boosters like Borneol, Frankincense, or Myrrh for additional therapeutic properties, those may be dissolved or emulsified within the gel. The ability to combine various TCM or conventional herbal components in the same patch underscores the flexible design. The user's personal comfort, the extent of muscle tension they experience, and the desired effect on wrinkling or pain relief can all be balanced by adjusting the patch composition and ridge arrangement.

In certain aspects, the final shape and orientation of patch 810 may differ from that strictly shown in FIG. 8, but the fundamental concept remains: there is a non-woven fabric backing, a second PET or reinforcement layer forming support ridges 820, and an optional third adhesive layer that may contain herbal extracts, analgesics, or remain herb-free for mechanical support only. A peelable release paper covers the adhesive layer until application, preserving sterility and preventing accidental adhesion. The user then positions patch 810 and experiences either cosmetic, therapeutic, or mechanical benefits, or some combination thereof.

In other aspects, the usage of patch 810 can be integrated into a regimen for alleviating muscle pain or limiting certain repetitive facial expressions. Because the ridges 820 can hamper movement slightly, individuals may find it easier to refrain from frowning, squinting, or other expressions that lead to deep lines. Meanwhile, if the patch 810 is loaded with anti-inflammatory or blood-circulation-promoting herbs, they may observe improvements in local tissue health over extended usage. In some aspects, the disclosed patch may be used to reduce the appearance of wrinkles, promote skin elasticity, or deliver analgesic relief to inflamed or overworked tissue.

In many implementations, the patch 810 suits a broad user base: from cosmetic consumers wishing to mitigate early wrinkling, to those needing mechanical bracing of a joint, to individuals seeking TCM-based analgesia for short- or long-term wear. FIG. 8 indicates the patch's general rectangular outline with vertical ridges, but also remains open-ended enough to represent alternative geometry (such as half-moon or bandage-style rectangular). The ability to incorporate different herbal solutions—cooling, warming, or none—further supports a wide range of medical, cosmetic, and protective applications.

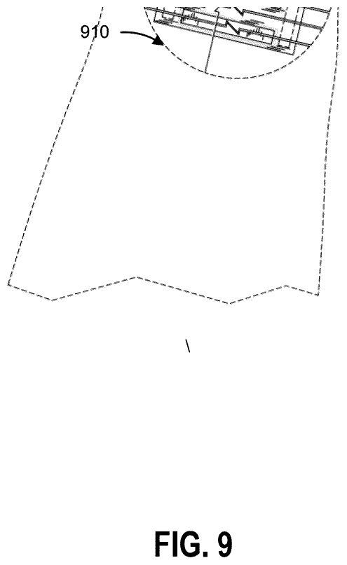

FIG. 9 illustrates an example of a representative placement of a clear mechanical-support patch.

In certain aspects, FIG. 9 illustrates a patch 910 positioned on a wrist 920, with multiple support ridges embedded or disposed across the surface to reduce excessive bending or unwanted joint movement. In many aspects, the patch 910 may include a first, non-woven, fabric backing layer or a transparent polymer film backing, a second layer composed of reinforcement strips, and a third adhesive layer that may or may not include herbal extracts, vitamins, or analgesic compounds. The illustrated wrist 920 demonstrates that while these patches have been described in some aspects for facial use, the same layered configuration may be adapted to other regions, such as elbows, knees, or wrists, depending on the final geometry cut or shape.

In some aspects, the patch 910 may be formed of a medical-grade aliphatic polyurethane (PU) film ranging in thickness from approximately 12 to 75 micrometers. This film may be blown or cast, then corona-treated and wound into a master roll for further processing. Alternatively, the backing could be a thermoplastic elastomer (TPE) film or an ethylene-vinyl acetate (EVA) sheet. If breathability is desired, the patch 910 may incorporate a spun-lace non-woven layer laminated to a clear polyethylene (PE) sheet, thereby yielding a backing with both transparency and moisture-vapor-permeable properties reminiscent of commercial dressings like Tegaderm™ or Nexcare®. In various aspects, the patch may also have micro-perforations to enhance air circulation near the skin, supporting multi-day wear with minimal irritation or sweat accumulation on the wrist 920.

In other aspects, the patch 910 may incorporate a skin-facing adhesive layer that could comprise a soft silicone gel, a low-tack acrylic pressure-sensitive adhesive (PSA), or a drug-in-adhesive matrix containing certain herbal or analgesic elements. For example, a silicone gel such as 3M 2476P might be used to ensure hypoallergenic performance with repositionability. In many aspects, the release liner that covers this adhesive prior to use may be formed from PET, polypropylene, or glassine paper with a fluorosilicone surface, enabling easy peel tabs. Once the user peels away this release liner, the patch 910 can be pressed against the wrist 920. Because the patch may be pre-cut into different shapes, a rectangular or half-moon design is equally possible, depending on user preference or anatomical location.

In some aspects, the patch 910 includes reinforcing support ridges (not labeled in the figure as new reference numbers, but the figure overview clarifies them as support ridges 930). These ridges may be formed of a variety of rigid to mid-modulus materials, such as cyclic olefin copolymer (COC), polycarbonate (PC), polymethyl methacrylate (PMMA), thermoplastic polyurethane (TPU), or polypropylene (PP) random copolymer. If a compostable or bio-based approach is preferred, oriented polylactic acid (PLA) or polycaprolactone (PCL) blends may be used. The ridges 930 may be laminated between the layer of polyurethane film skins, insert molded with silicone gel, co-extruded as a stiff core with flexible skins (such as 20 micrometer COC spine co-extruded between 10 micrometer EVA layers), or even 3D-printed in anisotropic lattice patterns. In many aspects, these ridges 930 may oppose muscle pulling forces that occur either on a face, wrist, or any other targeted region. As shown in FIG. 9, the ridges are oriented horizontally to brace the user's wrist 920, but in other scenarios they could be angled or placed vertically to address different vectors of motion.

In certain aspects, the patch 910 may also possess a third adhesive layer that includes an herbal infusion. If the user selects a mixture of Poria cocos mushroom, Rhizoma Dioscorea, and other traditional Chinese herbs, the patch might be used to deliver an anti-inflammatory or blood-flow-promoting effect in areas prone to repetitive stress. In alternative aspects, the adhesive layer might be replaced or supplemented by a cooling analgesic blend (Embodiment 1), referencing a classical San huang ointment formula with Scutellaria baicalensis, Coptis chinensis, and Phellodendron amurense. Menthol or Lygodium japonicum might be added to create a soothing, anti-inflammatory environment around the wrist 920, beneficial for mild tendonitis or swelling.

In many aspects, if a user desires a warming analgesic patch (Embodiment 2), the mixture in the adhesive may contain Angelica sinensis (Danggui), Corydalis yanhusuo (Yanhusuo), or Camphor, with the aim of relaxing tendons, alleviating muscle stiffness, and promoting blood circulation. This approach may be especially valued if the wrist region experiences chronic pain from repetitive tasks. The stiff ridges 930 integrated into the patch 910 may further limit wrist extension or flexion to relieve strain. In some aspects, the user might incorporate additional boosters, such as Safflower (Carthamus tinctorius, Honghua) or Salvia miltiorrhiza (Danshen), to bolster the warming effect and add mild analgesic properties.

In certain aspects, the patch 910 may remain completely free of herbal extracts, representing a Clear Mechanical-Support Patch. In this scenario, a medical-grade adhesive is placed on a transparent film that holds PET or similarly robust strips. The resultant patch could be nearly invisible, making it suitable for discreet daytime usage on the wrist, brow, or other body zones where reducing motion is helpful. The figure outlines that the patch 910, featuring support ridges 930, is adhered to the wrist 920. The same structural approach may be similarly leveraged for anti-wrinkle facial patches if these ridges are sized or oriented differently. Because the user can choose an herb-free version or a version loaded with analgesic or cosmetic agents, the system remains highly adaptable to multiple contexts—relieving muscle tension, delivering herbal extracts, or preventing frown lines.

In several aspects, the final patch 910 showcased in FIG. 9 may be manufactured in a continuous roll-to-roll process. For example, a thin polyurethane film may be extruded to form the backing layer, chilled, and corona-treated to promote adhesion. A slot-coating step may deposit a soft silicone adhesive, optionally infused with micro-encapsulated menthol or other botanical materials, at a coating weight of around 20-35 grams per square meter. Next, prefabricated COC or PC ribs could be laminated at a particular pitch using a heated nip at approximately 55° C. If breathability is crucial for multi-day wear, the manufacturer may laser-score small vents (say, 50 micrometers in diameter) through the backing but not fully through the adhesive, preserving a water barrier while allowing moisture vapor to escape. In other variations, a 3-axis fused deposition modeling (FDM) printing step might deposit polylactic acid lattices directly onto a tacky adhesive layer, culminating in an anisotropic pattern of ridges that match the user's typical muscle movement. Following that, the entire patch may be die-cut into the chosen geometry for a typical wrist coverage shape, culminating in the application of a PET or PP release liner. Alternatively, if inserting the patch into a silicone gel mold proves beneficial, a user might choose an insert-molding approach to overmold TPU ribs while sealing the edges of the patch.

In many aspects, the synergy of the mechanical approach (reinforcing members) and the optional herbal or vitamin-laden adhesive aims to reduce the discomfort or injury risk from everyday motions. If the user experiences pain or micro-tearing in the wrist 920, a patch with warming herbs might help stimulate local blood flow, while the ridges 930 reduce flexion. Conversely, if the user is dealing with inflammation or mild arthritis, a patch containing cooling botanicals such as Menthol, Camphor, or extracts from the classical San huang formula might help alleviate that discomfort as the patch is worn. Meanwhile, the same structural underpinnings remain consistent: a first polymer or non-woven backing, a second layer of reinforcement strips or PET ribs, and a third adhesive layer that could incorporate herbal extracts, analgesics, or remain transparent and herb-free.

In certain aspects, the flexible backing of patch 910 may be shaped to an intermediate curvature that closely follows the contours of the wrist 920, ensuring that the ridges 930 lie flush against the region requiring support. The patch edges may feature half-moon contours or rectangular corners, depending on user preference. If the user is concerned about sweat or water exposure, the patch might be water- and microbe-resistant, akin to advanced wound dressings. Because the patch may be configured for multi-day wear, the outer surfaces could permit moisture vapor to escape at a high rate while preventing infiltration of contaminants. The user might wear the patch overnight, which could be particularly beneficial if the adhesive is impregnated with a slow-release warming or cooling formula.

In other aspects, the support ridges 930 might be arranged in discrete sections rather than a single continuous strip, each section targeting a specific location on the wrist 920. If more thorough coverage is desired, the ridges could incorporate an anisotropic lattice that allows partial bending in one direction while restricting motion in another. For example, a user who frequently hyperextends the wrist may prefer these ridges to run parallel to the watchband area, or aligned with certain muscle groups that cause repeated motion. Because the figure highlights a small patch shape covering only part of the wrist, the system remains versatile enough to be upscaled for an elbow or knee if more robust coverage is needed.

In certain aspects, if the user desires synergistic cosmetic or collagen stimulation properties, the patch 910 might also be formulated with the same herbal extracts described for anti-wrinkle facial systems. The user could apply these on the wrist for dryness or early wrinkling. If vitamins A, B, D, E, and Zinc are introduced in the adhesive, they might further boost skin elasticity and healing rates. The patch shape in FIG. 9 primarily focuses on a mechanical support role but does not exclude layering in pro-collagen peptides or pro-skin-longevity compounds. Because the claims mention a mixture of herbal extracts and vitamins promoting skin elasticity and wrinkle reduction, this patch can be deployed for mild cosmetic improvements on the wrist or forearm, for example.

In many aspects, the ability to incorporate a peelable release paper onto the patch 910 ensures that the adhesive remains uncontaminated until application on the wrist 920. This arrangement may be especially helpful for users who want to keep multiple patches for repeated usage or for traveling. If an optional micro-perforated backing is chosen, the user might also find the patch comfortable enough for wearing during sports or daily errands without substantial sweat buildup. If a user has acute trauma, such as a mild sprain, a cooling analgesic approach may be selected, integrating Menthol or Lygodium japonicum for swelling reduction. Meanwhile, the ridges 930 physically discourage abrupt joint movements that might aggravate the injury. If the user has chronic stiffness, the warming analgesic approach (Angelica sinensis, Camphor, or Corydalis yanhusuo) might encourage blood flow over extended bouts of wear.

In several aspects, the user can remove the patch 910 after a certain period, typically 8-24 hours, by gently peeling off along the edges. If the adhesive is a soft silicone, the removal may be particularly gentle on sensitive or hair-bearing skin, reducing the likelihood of irritation or pulling. If a more permanent or stronger acrylic adhesive is applied for heavier mechanical constraints, the user may expect a firmer bond with the skin. The final choice depends on whether the user primarily seeks muscle or joint restriction, analgesic release, or mild cosmetic improvement on the wrist. In some aspects, a patch with a TPE film backing could be especially conformable, bending around radial or ulnar protrusions at the wrist without losing contact. Meanwhile, the ridges might remain stable, limiting the range of motion specifically in the direction that aggravates the user's condition.

In other aspects, the patch 910 can be part of a kit that includes differently sized patches for the same wrist or for related tasks. A narrower patch might be placed near the base of the thumb if that area experiences tension, while a larger one with more ridges could circle closer to the forearm. Some aspects might even combine cooling at certain ridges with warming in other segments, though more typical usage is a single formula per patch. Because the claims allow for replacements or supplements with different formulations, the user can pick whichever analgesic or cosmetic solution best suits them.

In certain aspects, the rotating or twisting motions of the wrist may be partially offset by angled ridges 930. FIG. 9 only shows a straightforward horizontal layout, yet the same design logic can apply for diagonal or cross-lattice patterns. If the user is a manual laborer or a gamer who repeatedly triggers tendon strain, the patch might reduce that motion to a tolerable range. If the user only wants mild tension relief or is focusing on relieving wrinkles in the wrist area, the ridges might be narrower or thinner. Because the wide range of materials includes everything from high-modulus COC for strong bracing to mid-modulus TPU or eco-friendly PLA for moderate stabilization, the patch can be customized for differing user profiles.

In many aspects, this single patch 910 design, when integrated with the claims describing a first, non-woven, fabric backing layer, a second layer of PET or other reinforcement strips, and a third adhesive layer containing herbal extracts, may unify mechanical and transdermal therapy for a robust solution. The potential for micro-perforations, warming or cooling herbal blends, and various optional boosters such as Borneol or Safflower align well with the broad categories enumerated by the claims. The figure underscores that non-facial uses—like the wrist in FIG. 9—are covered by the same structural logic. The user simply selects an appropriate shape, adhesive or herbal mixture, and reinforcement strip orientation for the area in question.

In certain implementations, if the patch must remain in place for multi-day intervals, the substrate might be gamma-sterilizable or produced in a clean environment, ensuring safe adhesion over an extended timeframe. If desired, a specialized timer release of botanicals could be achieved by micro-encapsulating them in a hydrophilic macromolecule gel matrix that slowly diffuses the active agents into the skin. This approach suits those who experience chronic pain or want consistent prophylactic measures against re-injury. The ridges 930 stand as the second layer that structurally maintains reduced muscle contraction, while the sticky herbal mixture or mechanical adhesive rests against the skin.

In other aspects, a user might combine the patch 910 with standard bandage-style usage or with additional bracing if more rigid immobilization is required. The integrated approach remains beneficial, as it can seamlessly deliver analgesic or cosmetic effects while providing partial mechanical support. Because each claim referenced contemplates both a transdermal effect and a mechanical effect, FIG. 9 highlights a direct representation of how these two functionalities intersect on a typical wrist scenario. If the user specifically wants only mechanical tension relief, they might choose the herb-free clear mechanical support patch. If they want a warming effect, they might choose a composition with Camphor or Cinnamon twig. If they aim for cooling, Menthol or Lygodium japonicum might be favored.

In several aspects, the patch 910 as shown in FIG. 9 is simply a partial illustration of how the layered structure adheres to a region not limited to the face. Although claims specifically address facial wrinkles, the same pattern of layering—backing, reinforcement, adhesive with or without herbs—may be shape-fitted for wrists, knees, or other body parts for non-facial usage. Indeed, the patch design may be scaled up or down. If a user selects a half-moon shape to wrap around the radial side of the wrist, both the backing film and ridges can be contoured accordingly. If a user wants a rectangular band large enough to cover the entire underside of the forearm near the wrist, the system remains quite feasible, leveraging the same fundamental manufacturing steps.

In many aspects, the described patch 910 and support ridges 930 on the user's wrist 920 thus demonstrate a primary example of the broad adaptability of the layered patch approach. By carefully choosing the backing material (PU, TPE, EVA, or layered non-woven laminates), the orientation and composition of reinforcement strips (COC, PC, PMMA, TPU, PLA, etc.), and the possible herbal or analgesic infusion, a wide range of functional or cosmetic targets can be addressed. The user may employ a single patch for mild daily tasks or multiple patches across different joints if more comprehensive support or analgesic coverage is needed. Because each claim contemplates that the mixture of herbal extracts and vitamins may be replaced or supplemented by alternative formulations (cooling, warming, or mechanical only), FIG. 9 confirms that these patches reconcile mechanical bracing with potential transdermal therapy across various body regions—particularly the wrist.

### CONCLUSION

While a particular form of the invention has been illustrated and described, it will be apparent that various modifications can be made without departing from the spirit and scope of the invention. For example, the combination of natural ingredients may vary from embodiment to embodiment, as may the proportions of each. Accordingly, it is not intended that the invention be limited, except as by the appended claims.

Particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific embodiments disclosed in the specification, unless the above Detailed Description section explicitly defines such terms. Accordingly, the actual scope of the invention encompasses not only the disclosed embodiments, but also all equivalent ways of practicing or implementing the invention.

The above detailed description of the embodiments of the invention is not intended to be exhaustive or to limit the invention to the precise form disclosed above or to the particular field of usage mentioned in this disclosure. While specific embodiments of, and examples for, the invention are described above for illustrative purposes, various equivalent modifications are possible within the scope of the invention, as those skilled in the relevant art will recognize. Also, the teachings of the invention provided herein can be applied to other systems, not necessarily the system described above. The elements and acts of the various embodiments described above can be combined to provide further embodiments.

All of the above patents and applications and other references, including any that may be listed in accompanying filing papers, are incorporated herein by reference. Aspects of the invention can be modified, if necessary, to employ the systems, functions, and concepts of the various references described above to provide yet further embodiments of the invention.

Changes can be made to the invention in light of the above Detailed Description. While the above description details certain embodiments of the invention and describes the best mode contemplated, no matter how detailed the above appears in text, the invention can be practiced in many ways. Therefore, implementation details may vary considerably while still being encompassed by the invention disclosed herein. As noted above, particular terminology used when describing certain features or aspects of the invention should not be taken to imply that the terminology is being redefined herein to be restricted to any specific characteristics, features, or aspects of the invention with which that terminology is associated.

While certain aspects of the invention are presented below in certain claim forms, the inventor contemplates the various aspects of the invention in any number of claim forms. Accordingly, the inventor reserves the right to add additional claims after filing the application to pursue such additional claim forms for other aspects of the invention.

Clause 1. A dermal patch, comprising: a first, non-woven, fabric backing layer; a second layer of polyethylene terephthalate (PET) or reinforcement strips disposed on the backing layer; a third adhesive layer disposed over the layer of PET or reinforcement strips and the backing layer, comprising a mixture of herbal extracts and vitamins, including Poria cocos mushroom, Radix et Rhizoma salviae Miltiorrhizae, Rhizoma dioscorea, Polygonum cuspidatum, Angelica dahurica, Amygdalus communis Vas, Radix et Rhizoma Notogiseng, and Rhizoma ginseng, applied over said layer of PET or other reinforcement strips; and a peelable release paper disposed on said adhesive layer, wherein the adhesive layer is formulated to promote transdermal delivery of said herbal extracts and vitamins to improve skin elasticity and reduce an appearance of wrinkles and fine lines when applied to the skin.

Clause 2. The dermal patch of clause 1, wherein the herbal extracts and vitamins are present in the mixture, by weight, in an amount of approximately 64-96% purified water, 4.8-6% Poria cocos mushroom, 0.8-2.4% Radix et Rhizoma salviae Miltiorrhizae, 4.8-6% Rhizoma Dioscorea, 0.8-4% Polygonum cuspidatum, 0.8-4% Angelica dahurica, 0.8-4% Amygdalus communis Vas, 0.4-1.6% Radix et Rhizoma Notogiseng, and 0.4-1.6% Rhizoma ginseng.

Clause 3. The dermal patch of clause 1, wherein said mixture of herbal extracts and vitamins are naturally sticky and contribute to an adhesive property of the first layer.

Clause 4. The dermal patch of clause 1, wherein said PET or other reinforcement strips inhibit excessive muscle contractions when the patch is applied to the skin.

Clause 5. The dermal patch of clause 1 wherein the mixture of herbal extracts and vitamins further comprises antioxidants vitamins A, B, D, and E, as well as Zinc.

Clause 6. The dermal patch of clause 1, wherein the herbal extracts further provide anti-inflammatory benefits and promote blood flow.

Clause 7. The dermal patch of clause 1, wherein the adhesive layer is specifically contoured to conform to various areas of a human face.

Clause 8. The dermal patch of clause 1, pre-cut into shapes specifically contoured to conform to various targeted areas of a human face, such as around eyes, forehead, or mouth, each shape designed to maximize skin contact and an overall transdermal delivery system effectiveness in those areas.

Clause 9. The dermal patch of clause 1, wherein the mixture of herbal extracts and vitamins is replaced or supplemented by an alternative formulation selected from a cooling analgesic blend comprising one or more menthol-type or traditional Chinese medicine (TCM) cooling herbs configured to alleviate acute trauma or inflammatory flare-ups; a warming analgesic blend comprising one or more cinnamon-type or TCM warming herbs configured to stimulate blood flow for chronic conditions; or an herb-free formulation in which the adhesive layer remains medical-grade but transparent, configured primarily for mechanical support with minimal visibility, wherein a core structure of a non-woven fabric and reinforcement strips remains consistent so as to limit undesired muscle or joint movement and support transdermal contact (if applicable) over a targeted region.

Clause 10. The dermal patch of clause 1, further configured in a bandage-style rectangular or half-moon shape that is sized to accommodate application to at least one non-facial region selected from a wrist, knee, back, or elbow, wherein the reinforcement strips include polyethylene terephthalate (PET) ribs dimensioned to reduce joint overstretching or excessive flexion while the patch is worn.

Clause 11. The dermal patch of clause 1, wherein the patch is optionally constructed with a micro-perforated backing for extended hour or multi-day wear, one or more perforations enabling moisture evaporation and improved air circulation while maintaining either cooling analgesic delivery for acute pain relief; warming analgesic delivery for chronic conditions; or transparent mechanical support to limit undesired motion.

Clause 12. A dermal patch for transdermal nutrient application, comprising: a base structure including a non-woven fabric backing and reinforcement strips; a therapeutically effective amount of a herbal and vitamin composition for promoting muscle regrowth, collagen regeneration, and skin repair; and a contoured design shaped to maintain continuous skin contact for sustained herb and vitamin absorption, wherein the dermal patch is specifically designed to deliver targeted remedial agents to facial regions prone to wrinkles and fine lines over an extended period.

Clause 13. The dermal patch of clause 12, wherein a herbal and vitamin composition includes Poria cocos mushroom, Radix et Rhizoma salviae Miltiorrhizae, Rhizoma Dioscorea, and additional herbal ingredients known for their skin-enhancing properties.

Clause 14. The dermal patch of clause 12, wherein the reinforcement strips are composed of PET material that provides structural integrity to the facial patch and assists in a reduction of muscle contraction.

Clause 15. The dermal patch of clause 12, further comprising a release paper enabling secure storage and easy application of the facial patch.

Clause 16. A method for manufacturing a dermal patch for reducing an appearance of wrinkles and fine lines, comprising: preparing a substrate layer by affixing reinforcement strips onto a non-woven fabric backing; creating an adhesive layer by dissolving a composition of Poria cocos mushroom, Radix et Rhizoma salviae Miltiorrhizae, Rhizoma Dioscorea, Angelica dahurica, Amygdalus communis Vas, Radix et Rhizoma Notogiseng, and Rhizoma ginseng, and vitamins in purified water heated to a boil, to form a naturally sticky solution; applying the adhesive layer containing the naturally sticky solution onto the prepared substrate layer with the reinforcement strips; and covering the adhesive layer with a peelable release paper, configured for secure storage and facilitating easy application of the dermal patch to human facial skin, wherein the dermal patch is designed to facilitate transdermal delivery of a herbal and vitamin composition, thereby improving skin elasticity and reducing the appearance of wrinkles and fine lines over an extended period.

Clause 17. The method of clause 16, wherein the composition further includes anti-inflammatory agents and agents to promote blood flow, specifically designed to form a barrier to retain moisture in the skin, enhancing an effectiveness of the adhesive layer in delivering these components transdermally.

Clause 18. The method of clause 16, wherein the sticky solution comprises a specific ratio of ingredients which is conducive to transdermal delivery efficiency; and the specific ratio is achieved by dissolving the composition in purified water at a volume ratio of approximately 40-90% water to a total weight of the adhesive layer, with the composition proportionately adjusted to their therapeutic concentrations.

Clause 19. The method of clause 16, comprising designing the adhesive facial patch to prevent excessive muscle contraction, wherein the reinforcement strips are strategically placed within the substrate layer to distribute the composition evenly during application and optimize effectiveness of a transdermal delivery system.

Clause 20. The method of clause 16, upon manufacture further comprising: performing a regimen for facial skin rejuvenation comprising securing a facial patch, manufactured according to the method specified in claim 16, to an area of the skin susceptible to wrinkles; and ensuring the facial patch remains attached for an overnight period or for at least 20 minutes, to promote an extended release and skin penetration of herbal extracts and vitamins causing skin health improvement and wrinkle reduction, wherein this extended release is facilitated by a unique composition of the adhesive layer and deliberate design of a reinforcement structure of the facial patch.

What is claimed is:

1. A dermal patch, comprising:
a first, non-woven, fabric backing layer;
a second layer of polyethylene terephthalate (PET) or reinforcement strips disposed on the backing layer;
a third adhesive layer disposed over the layer of PET or reinforcement strips and the backing layer, comprising a mixture of herbal extracts and vitamins, including Poria cocos mushroom, Radix et Rhizoma salviae Miltiorrhizae, Rhizoma dioscorea, Polygonum cuspidatum, Angelica dahurica, Amygdalus communis Vas, Radix et Rhizoma Notogiseng, and Rhizoma ginseng, applied over said layer of PET or other reinforcement strips; and
a peelable release paper disposed on said adhesive layer, wherein the adhesive layer is formulated to promote transdermal delivery of said herbal extracts and vitamins to improve skin elasticity and reduce an appearance of wrinkles and fine lines when applied to the skin.

2. The dermal patch of claim 1, wherein the herbal extracts and vitamins are present in the mixture, by weight, in an amount of approximately 64-96% purified water, 4.8-6% Poria cocos mushroom, 0.8-2.4% Radix et Rhizoma salviae Miltiorrhizae, 4.8-6% Rhizoma dioscorea, 0.8-4% Polygonum cuspidatum, 0.8-4% Angelica dahurica, 0.8-4% Amygdalus communis Vas, 0.4-1.6% Radix et Rhizoma Notogiseng, and 0.4-1.6% Rhizoma ginseng.

3. The dermal patch of claim 1, wherein said mixture of herbal extracts and vitamins are naturally sticky and contribute to an adhesive property of the first layer.

4. The dermal patch of claim 1, wherein said PET or other reinforcement strips inhibit excessive muscle contractions when the patch is applied to the skin.

5. The dermal patch of claim 1 wherein the mixture of herbal extracts and vitamins further comprises antioxidants vitamins A, B, D, and E, as well as Zinc.

6. The dermal patch of claim 1, wherein the herbal extracts further provide anti-inflammatory benefits and promote blood flow.

7. The dermal patch of claim 1, wherein the adhesive layer is specifically contoured to conform to various areas of a human face.

8. The dermal patch of claim 1, further comprising pre-cut into shapes specifically contoured to conform to various targeted areas of a human face, such as around eyes, forehead, or mouth, each shape designed to maximize skin contact and an overall transdermal delivery system effectiveness in those areas.

9. The dermal patch of claim 1, wherein the mixture of herbal extracts and vitamins is replaced or supplemented by an alternative formulation selected from a cooling analgesic blend comprising one or more menthol-type or traditional Chinese medicine (TCM) cooling herbs configured to alleviate acute trauma or inflammatory flare-ups; a warming analgesic blend comprising one or more cinnamon-type or TCM warming herbs configured to stimulate blood flow for chronic conditions; or an herb-free formulation in which the adhesive layer remains medical-grade but transparent, configured primarily for mechanical support with minimal visibility, wherein a core structure of a non-woven fabric and reinforcement strips remains consistent so as to limit undesired muscle or joint movement and support transdermal contact over a targeted region.

10. The dermal patch of claim 1, further configured in a bandage-style rectangular or half-moon shape that is sized to accommodate application to at least one non-facial region selected from a wrist, knee, back, or elbow, wherein the reinforcement strips include polyethylene terephthalate (PET) ribs dimensioned to reduce joint overstretching or excessive flexion while the patch is worn.

11. The dermal patch of claim 1, wherein the patch is optionally constructed with a micro-perforated backing for extended hour or multi-day wear, one or more perforations enabling moisture evaporation and improved air circulation while maintaining either cooling analgesic delivery; warming analgesic delivery; or transparent mechanical support to limit undesired motion.

12. A dermal patch for transdermal nutrient application, comprising:
a base structure including a non-woven fabric backing and reinforcement strips;
a therapeutically effective amount of a herbal and vitamin composition for promoting muscle regrowth, collagen regeneration, and skin repair; and
a contoured design shaped to maintain continuous skin contact for sustained herb and vitamin absorption, wherein the dermal patch is specifically designed to deliver targeted remedial agents to facial regions prone to wrinkles and fine lines over an extended period.

13. The dermal patch of claim 12, wherein a herbal and vitamin composition includes Poria cocos mushroom, Radix et Rhizoma salviae Miltiorrhizae, Rhizoma dioscorea, and additional herbal ingredients known for their skin-enhancing properties.

14. The dermal patch of claim 12, wherein the reinforcement strips are composed of PET material that provides structural integrity to the facial patch and assists in a reduction of muscle contraction.

15. The dermal patch of claim 12, further comprising a release paper enabling secure storage and easy application of the facial patch.

16. A method for manufacturing a dermal patch for reducing an appearance of wrinkles and fine lines, comprising:
preparing a substrate layer by affixing reinforcement strips onto a non-woven fabric backing;
creating an adhesive layer by dissolving a composition of Poria cocos mushroom, Radix et Rhizoma salviae Miltiorrhizae, Rhizoma dioscorea, Angelica dahurica, Amygdalus communis Vas, Radix et Rhizoma Notogiseng, and Rhizoma ginseng, and vitamins in purified water heated to a boil, to form a naturally sticky solution;
applying the adhesive layer containing the naturally sticky solution onto the prepared substrate layer with the reinforcement strips; and
covering the adhesive layer with a peelable release paper, configured for secure storage and facilitating easy application of the dermal patch to human facial skin, wherein the dermal patch is designed to facilitate transdermal delivery of a herbal and vitamin composition, thereby improving skin elasticity and reducing the appearance of wrinkles and fine lines over an extended period.

17. The method of claim 16, wherein the composition further includes anti-inflammatory agents and agents to promote blood flow, specifically designed to form a barrier to retain moisture in the skin, enhancing an effectiveness of the adhesive layer in delivering these components transdermally.

18. The method of claim 16, wherein the sticky solution comprises a specific ratio of ingredients which is conducive to transdermal delivery efficiency; and
the specific ratio is achieved by dissolving the composition in purified water at a volume ratio of approximately 40-90% water to a total weight of the adhesive layer, with the composition proportionately adjusted to their therapeutic concentrations.

19. The method of claim 16, further comprising designing the adhesive facial patch to prevent excessive muscle contraction, wherein the reinforcement strips are strategically placed within the substrate layer to distribute the composition evenly during application and optimize effectiveness of a transdermal delivery system.

20. The method of claim 16, upon manufacture further comprising:
performing a regimen for facial skin rejuvenation comprising securing a facial patch to an area of the skin susceptible to wrinkles; and
ensuring the facial patch remains attached for an overnight period or for at least 20 minutes, to promote an extended release and skin penetration of herbal extracts and vitamins causing skin health improvement and wrinkle reduction, wherein this release is facilitated by a unique composition of the adhesive layer and deliberate design of a reinforcement structure of the facial patch.
